(19) United States
(12) Patent Application Publication
(10) Pub. No.: US 2019/0298840 A1
(43) Pub. Date: Oct. 3, 2019

(54) FORMULATIONS COMPRISING HETEROCYCLIC RING SYSTEMS AND USES THEREOF

(71) Applicant: Particle Sciences, Inc., Bethlehem, PA (US)

(72) Inventors: Mark A. Mitchnick, East Hampton, NY (US); Bob Becker, Nazareth, MA (US); Bruce Frank, Pennington, NJ (US); Victoria Cofre, Quakertown, PA (US)

(21) Appl. No.: 16/272,570

(22) Filed: Feb. 11, 2019

Related U.S. Application Data
(63) Continuation of application No. PCT/US17/49451, filed on Aug. 30, 2017.
(60) Provisional application No. 62/381,134, filed on Aug. 30, 2016.

(51) Int. Cl. A61K 47/54; G01N 33/58; G01N 33/53; A61K 47/64; A61K 47/68

(52) U.S. Cl. CPC A61K 47/545; G01N 33/582; A61K 47/557; A61K 47/643; A61K 47/6835; G01N 33/5306

(57) ABSTRACT

The present invention relates to liquid compositions comprising heterocyclic ring systems that interact with biological molecules through non-covalent interactions. The non-covalent interactions between heterocyclic rings and biological molecules comprise interactions ranging from electrostatic interactions, hydrogen bond interactions, van der Waals interactions, and hydrophobic interactions.

### CROSS REFERENCE TO RELATED APPLICATIONS

This PCT application claims priority to U.S. Provisional Application 62/381,134 filed on Aug. 30, 2016. The teachings of the aforesaid provisional application are incorporated herein by reference in its entirety.

### TECHNICAL FIELD

The field of the invention relates to liquid compositions comprising heterocyclic ring systems.

### BACKGROUND

The linkage of macromolecules such as proteins and nucleic acids to other chemical moieties and/or to surfaces has become an important aspect of biopharmaceutical, vaccine and diagnostic development and manufacturing. These linkages are used to associate proteins to the surfaces of structures like those used in diagnostics devices, drug delivery devices and medical devices and/or to link molecules together, for examples: the PEGylation of proteins, the antibody-drug conjugates, the labeling of proteins (including antibodies and antigens) with enzymatic or florescent markers or biotin (for forming avidin complexes) and the production of carrier protein-polysaccharide conjugates for vaccine development. These linkages have typically used covalent bonds using a chemical, water soluble, conjugation linkers that react with the chemical entities to be linked forming covalent bonds with both entities. These bonds are considered relatively stable in biological systems but their use in the development and manufacturing of biopharmaceutical products is complicated by the fact that they irreversibly, chemically modify/alter the proteins being conjugated.

Alternatively, electrostatic bonding has been successfully used to associate different chemical entities in biopharmaceutical and pharmaceutical formulations. Electrostatic bonding does not chemically modify proteins that are being developed or formulated into products making this process simpler and more commercially scalable. However, electrostatic bonding can be unstable in the presence of antichaotropic salts/ions (Queiroz, Tomaz and Cabral, Hydrophobic interaction chromatography of proteins, J Biotechnol. 2001 May 4; 87(2):143-59); (Pahlman, Rosengren and Hjerten Hydrophobic interaction chromatography on uncharged Sepharose derivatives. Effects of neutral salts on the adsorption of proteins, J Chromatogr. 1977 Jan. 21; 131:99-108.) like PO4³⁻, SO42⁻, and NH₄⁺, which are prevalent in biological fluids. It is often desired that the linkages between the chemical entities be stable upon administration in patients, therefore the use of electrostatic bonding in these applications has limitations. Further, electrostatic bonding is dependent on the charges of the two entities being bound being opposite and of sufficient magnitude, a condition that is often difficult to achieve under physiologic pH.

### SUMMARY OF THE EMBODIMENTS

One embodiment of the invention relates to a composition comprising a heterocyclic ring, a synthetic stem, and a biological molecule wherein the heterocyclic ring is (a) covalently linked to the synthetic stem and (b) non-covalently associated with the biological molecule.

In one embodiment of the invention, the non-covalent association is stable upon exposure to antichaotropic salts.

In one embodiment of the invention, the non-covalent association comprises hydrophobic interactions.

In one embodiment of the invention, the hydrophobic interactions are selected from the group consisting of pi interactions, pi-pi interactions and pi stacking interactions

In one embodiment of the invention, the biological molecules are non-covalently associated with a surface using a composition of the invention.

In another embodiment, the surface is not a particle surface.

In another embodiment, the surface is not a nanoparticle surface.

In one embodiment of the invention, the synthetic stem comprises a substituted or un-substituted hydrocarbon.

In one embodiment of the invention, the synthetic stem comprises a small molecule drug.

In one embodiment of the invention, the synthetic stem comprises poly ethylene glycol (PEG).

In one embodiment of the invention, the heterocyclic ring in the composition is water miscible.

In one embodiment of the invention, the heterocyclic ring comprises a heterocyclic aromatic quaternary amine.

In one embodiment of the invention, the heterocyclic ring comprises a pyridinium ring.

In one embodiment of the invention, the heterocyclic ring is positively charged over a pH range of about 3 to about 10.

In one embodiment of the invention, the biological molecule is selected from a group consisting of antibodies, proteins, peptides, DNA, RNA and DNA ligands.

In one embodiment of the invention relates to a composition comprising a heterocyclic ring, a synthetic stem, and a biological molecule wherein the heterocyclic ring is (a) covalently linked to the synthetic stem and (b) non-covalently associated with the biological molecule, wherein more than one copy of the heterocyclic ring is covalently linked to the synthetic stem.

In one embodiment of the invention, the heterocyclic rings are covalently linked to the synthetic stem and are non-covalently associated with the same or different biological molecules, linking multiple copies of the biological molecules to the same synthetic stem.

In one embodiment of the invention, the synthetic stem is covalently or non-covalently associated with molecules in a non-aqueous phase, and the heterocyclic rings are non-covalently associated with the same or different biological molecules in an aqueous phase.

In one embodiment of the invention, the non-covalent association is reversible or partially reversible under physiological conditions.

In one embodiment of the invention, the synthetic stem is covalently attached to said small molecule drug and said heterocyclic rings are non-covalently associated with the same or different biological molecules. The small molecule drug in one embodiment is used to treat diseases of oncology or immunology. The small molecule drug in one embodiment is used to treat diseases of infection or inflammation.

In one embodiment of the invention, the synthetic stem is covalently linked to a small molecule drug or a macromolecular drug, said heterocyclic rings are non-covalently associated to said biological molecules, wherein said biological molecules are of same kind or different from one another.

In one embodiment of the invention, the heterocyclic ring is a pyridinium ring system.

In one embodiment of the invention, the pyridinium ring is covalently attached to a small molecule drug and also hydrophobically binds to an antibody with desired specificity forming an Antibody-Drug-Complex (ADCom)

In another embodiment of the invention, the pyridinium ring is covalently attached to a small molecule drug and hydrophobically binds to biological protein or DNA or a ligand that binds to a receptor facilitating targeted delivery to cells or tissues.

In another embodiment of the invention, the pyridinium ring is covalently attached to a strand of PEG and pyridinium hydrophobically binds to a biopharmaceutical protein. The protein-pyridinium-PEG complex has a longer biological half-life than the protein alone when administered into animals.

In another embodiment of the invention, the synthetic stem is a hydrocarbon polymer stem to which multiple copies of pyridinium rings are covalently attached. The pyridinium rings bind to a biopharmaceutical forming a complex. The complex upon administration into animals gradually dissociates, since it is non-covalently bound, and provides a sustained release of the biopharmaceutical.

In another embodiment of the invention, the synthetic stem is a hydrocarbon polymer stem to which multiple copies of pyridinium rings are covalently attached. The pyridinium ring systems bind to a biopharmaceutical antibody with cell surface receptor specificity forming a complex. The complex upon administration into subject, binds to the cell surface receptors and efficiently cross-links receptors thereby better activating cells to respond to the stimulus.

In another embodiment of the invention, the synthetic stem is a hydrocarbon polymer stem to which multiple copies of pyridinium rings are covalently attached. The pyridinium ring systems binds to more than one biopharmaceutical or diagnostic reagent proteins forming a complex of the proteins. For example, antibody with desired specificity complexed with an enzyme bound to pyridinium constructs can be used for a colorimetric reaction in a diagnostic assay or in ELISA or other immunoassay diagnostics.

In one embodiment of the invention, pyridinium ring is covalently attached to a hydrocarbon stem to which multiple copies of a small drug is attached. The pyridinium—drug complex when mixed with a protein like albumin, improves the bioavailability and half-life of the drug formulation upon administration.

In one embodiment of the invention, pyridinium ring is covalently attached to a hydrocarbon stem to which multiple copies of diagnostic marker is attached. This pyridinium—diagnostic construct is mixed with a protein ligand or antibody with desired specificity to form a complex. The complex is then administered and ligand/antibody binds to the desired receptor/antigen targeting the marker for diagnostic analyses.

In one embodiment, pyridinium bound to a hydrocarbon stem is used to coat a plastic surface like an immunoassay plate, thereby coating pyridinium on the surface of a plate. The pyridinium coated surface can then be used to coat proteins by hydrophobic bounding for use in diagnostic assays.

### BRIEF DESCRIPTION OF THE DRAWINGS

Those skilled in the art should more fully appreciate advantages of various embodiments of the invention from the following “Detailed Description of Specific Embodiments,” discussed with reference to the drawings summarized immediately below.

The foregoing features of embodiments will be more readily understood by reference to the following detailed description, taken with reference to the accompanying drawings, in which:

FIGS. 1A-1F schematically show an examples of synthetic stem with heterocyclic ring systems. Note that the schematics are not drawn to scale. The icosahedron and dotted circle schematically represents two different types of biological molecules.

FIG. 2A shows pyridinium ring systems and fluorophore tethered to a synthetic stem.

FIG. 2B shows pyridinium ring systems conjugated with multiple copies of a biological molecule.

FIG. 3 shows pyridinium ring systems and chromophore tethered to a synthetic stem.

FIG. 4 shows pyridinium ring systems and biotin tethered to a synthetic stem.

FIG. 5 shows the synthetic scheme for producing pyridinium ring systems and fluorophore tethered to a synthetic stem

FIG. 6A shows the synthetic scheme for producing pyridinium ring systems and chromophore tethered to a synthetic stem.

FIG. 6B shows an embodiment wherein two different biological molecules (antibody and another different protein) are conjugated to the pyridinium ring systems.

FIG. 7 shows the synthetic scheme for producing pyridinium ring systems and biotin tethered to a synthetic stem.

FIG. 8 shows examples of heterocyclic systems that can be used to make compositions of the invention.

FIG. 9 shows examples of charged heterocyclic amines that can be used to make the compositions of the invention.

FIG. 10 shows the relationship between the concentration of heterocyclic ring compositions and the amount of bound IgG bound to the compositions.

FIG. 11 shows the synthetic scheme for producing PEGylated pyridinium ring systems.

FIGS. 12A-12D illustrate an embodiment of the invention for ELISA studies. (A) Antibodies that are specific for the antigen of interest are pre-mixed with a pyridinium-biotin construct. (B) the pyridinium binds non-covalently, hydrophobically with the antibody, labeling the antibody with biotin (this replaces the previous need to covalent link biotin to these antibodies). (C) biotin labeled antibodies are then added to an ELISA plate coated with the antigen for which the antibody is specific, and subsequently unbound antibodies are removed. (D) the remaining bound biotin labeled antibodies are detected and measured by having avidin-horse radish peroxidase (HRP) bind to the biotin, and after washing away unbound biotin-HRP, using substrates for HRP in a colorimetric reaction that will be measured in an ELISA reading instrument.

FIG. 13A illustrates multiple antibodies complexed with multiple pyridinium constructs. FIG. 13B illustrates antibodies gradually disassociating from pyridinium. FIGS. 13A-13B illustrate drug sustained release according to the invention, where one (or more) biological molecules are mixed with a construct composed of multiple pyridinium rings attached to a hydrocarbon chain (in this example). The binding of multiple biological molecules to a copy of the construct, and likewise several copies of the construct to binding a biological molecule ultimately results in the forming of a large complex which could be administered. Since the binding of pyridinium to the biological molecule is reversible, the administered complex can gradually dissociate releasing the biological molecule in a sustained release manner.

FIGS. 14A-14B illustrate application of the invention to chromatography where (A) a chromatography matrix that has multiple pyridinium rings (the heterocyclic aromatic ring in this example) is mixed with a biological molecule that will hydrophobically bind to the pyridinium (as shown in B). Subsequently unbound materials can be washed from the chromatography matrix and biological molecule could be eluted from the matrix using a chaotropic agent (a step not illustrated in this figure).

FIG. 15 illustrates drugs linked to antibodies by hydrophobic bonding with a pyridinium ring.

FIG. 16 illustrates binding affinity curves for Pyridinium-Fluorescein construct with IgG in presence of PBS and saline.

FIG. 17 illustrates binding affinity curves for Pyridinium-Fluorescein construct with human serum albumin (HSA) in presence of PBS and saline.

FIG. 18 illustrates the comparative binding affinity curves for pyridinium constructs with IgG and HSA under saline solution.

FIG. 19 illustrates the comparative binding affinity curves for pyridinium constructs with IgG and HSA under PBS solution.

FIG. 20 illustrates ELISA assay results for pyridinium constructs with IgG and BSA.

FIG. 21 illustrates periodate oxidation of dextran.

FIG. 22 illustrates reductive amination of dextran.

FIG. 23 illustrates hydrophobic bonding between a representative peptide vasopressin (Cys-Tyr-Phe-Gln-Asn-Cys-Pro-Arg-Gly) and pyridinium construct, with hydrophobic interactions between aromatic rings shown in hash lines.

FIG. 24 illustrates hydrophobic bonding between nucleic acid and pyridinium construct displaying hydrophobic base stacking and intercalation type interactions.

### DETAILED DESCRIPTION OF SPECIFIC EMBODIMENTS

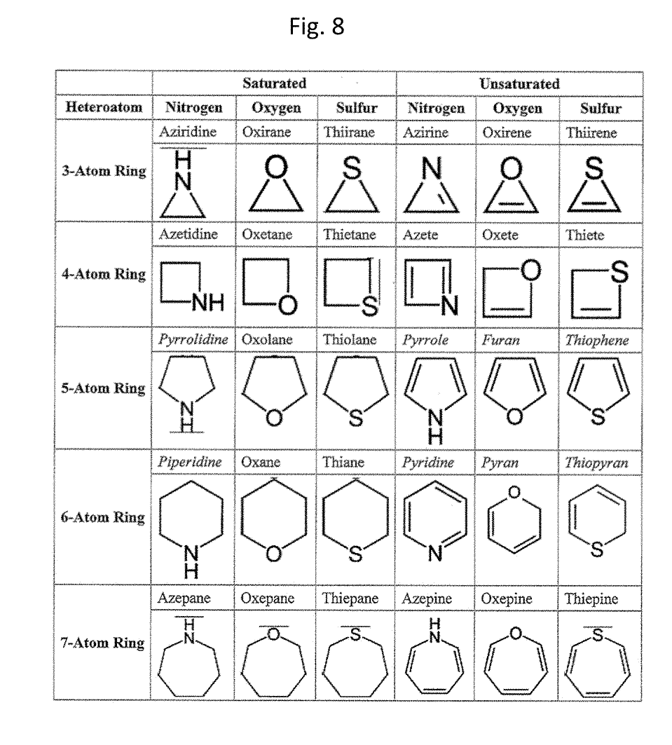

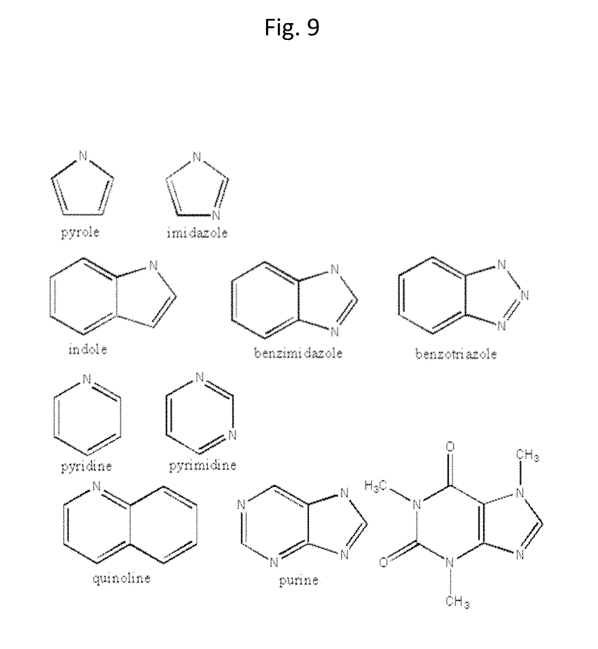

In one aspect of the invention, we use water miscible organic moieties that also have the capacity to from hydrophobic bonding with hydrophobic regions of proteins forming stable linkages between proteins and surfaces, like lipid bilayers, membranes or plastic surfaces present in multiwall plates or metallic surface of medical instruments, or other chemical entities like PEG and long-chain sugars (e.g. dextran) even in solutions containing antichaotropic salts. There are few chemical moieties that have the properties of having good water solubility or miscibility (greater than 104 mg/liter) and still prefer being in hydrophobic solvents as measured by Log P (having a Log P that is greater than 0). One non-limiting example of such compounds is heterocyclic aromatic compound pyridine. Other Heterocyclic aromatic amines and heterocyclic ring systems as shown in FIG. 9 can be covalently bond to other chemical moieties via the amine in the heterocyclic ring forming a quaternary amine, which fixes that amine into a positive charge that is independent of the pH of the aqueous solutions and hence remains water soluble. Similar interactions can be obtained through the heterocyclic atoms present in the heterocyclic ring systems such as oxygen and sulphur. Examples of various heterocyclic compounds that can be used in place of pyridinium compounds are show in FIG. 8.

Other organic compounds with the comparable characteristics include other heterocyclic aromatic compounds like pyrole, pyran, oxolane, and heterocyclic non-aromatic compounds like piperdine, oxan, oxolone, thietane, and thiirane. Other non-heterocyclic organic compounds with these properties include: phenol, 2-butoxyethanol, butyric acid, dimethloxyethane, furfuryl alcohol, 1-propanol, 2-propanol, and propanoic acid.

The invention is based on the unexpected discovery that certain compounds exhibiting properties of being water soluble surprisingly seem to prefer hydrophobic solvents, like that of pyridinium as well. These compounds form stable hydrophobic bonds with proteins. The invention is useful to stably link proteins to surfaces and other chemical entities. The linkages are stable in antichaotropic salts solutions, which would otherwise destabilize ionic bonds. The invention has a broad range of applications especially in the development of biopharmaceutical, pharmaceutical, vaccine and diagnostic human and veterinary products.

### Definitions

As used in this description and the accompanying claims, the following terms shall have the meanings indicated, unless the context otherwise requires:

“Synthetic stem”, as used herein, refers to a hydrocarbon chain consisting of more than one carbon; for example, 2, 4, 6, 8, 10, 20, 40 or 50 carbon atoms. The carbon atoms on the synthetic stem are optionally substituted with halides, oxygen, nitrogen, sulphur, phosphorous or a combination thereof. The synthetic stem or portions thereof can be either generated by synthetic chemistry, like that of many small molecule drugs, or biologically produced, like that for natural polymers including sugars/polysaccharides, and then covalently attached to the heterocyclic ring by chemical reactions and/or linkers.

A “Heterocyclic ring”, as used herein, is a cyclic compound that has atoms of at least two different elements as members of its ring. Preferably, the different elements are selected from nitrogen, oxygen, sulphur and combinations thereof. The compound is cyclic by virtue of its forming a ring and, therefore, it will include at least four atoms, and may include 5, 6, 7, 8, or more atoms.

A “Biological molecule”, as used herein, refers to a molecule that is produced by a biological process, in living organisms, in vitro biological processes, or synthetic in vitro processes that can be used to replace a natural biological process. Biological molecules include large macromolecules such as proteins, peptides, carbohydrates, lipids, and nucleic acids, as well as small molecules such as primary metabolites, secondary metabolites, and natural products.

“Electrostatic interactions”, as used herein, refer to interactions between and among cations and anions. Electrostatic interactions can be either attractive or repulsive, depending on the nature of the charged ions.

“Noncovalent interactions”, as used herein, refer to dispersed variations of electromagnetic interactions between molecules or within a molecule. Non-covalent interactions can be generally classified into five categories, electrostatic, pi-effects, van der Waals forces, hydrogen bonding and hydrophobic interactions.

“Hydrogen bond interactions”, as used herein, are types of attractive (dipole-dipole) interactions between an electronegative atom and a hydrogen atom bonded to another electronegative atom. A hydrogen bond interaction tends to be stronger than van der Waals forces, but weaker than covalent bonds or ionic bonds.

“van der Waals interactions”, as used herein, are interactions driven by induced electrical interactions between two or more atoms or molecules that are very close to each other. Van der Waals interaction is the weakest of all intermolecular attractions between molecules.

“Hydrophobic interactions”, as used herein, are entropy-driven interactions between uncharged substituents on different molecules without a sharing of electrons or protons.

“Antichaotropic salts”, as used herein, are molecules in an aqueous solution that increase the hydrophobic effects in the solution. Ammonium sulphate, sodium phosphate, ammonium citrate, sodium citrate, ammonium phosphate, sodium fluoride and ammonium fluoride are some non-limiting examples of antichaotropic salts/ions.

“Pi-pi interactions”, as used herein, is a type of non-covalent interaction that involves π systems. The electron-rich π system in heterocyclic ring or aromatic ring can interact with a metal (cationic or neutral), an anion, another molecule and even another π system. Non-covalent interactions involving π systems can be pivotal to biological events such as protein-ligand recognition.

“Aqueous phase”, as used herein, is the homogeneous part of a heterogeneous system that consists of water or a solution in water of a compound or a mixture of compounds.

“Non-aqueous phase”, as used herein, refers to solid phase where the cohesive force of matter is strong enough to keep the molecules or atoms in the given positions, restraining the thermal mobility.

“Small molecule drug”, as used herein, is a low molecular weight (preferably 10-100 Daltons, 100, 150, 250, 500, and <900 Daltons) organic compound that may affect, alter or block biological processes inside cells, tissues and living organisms.

“Macromolecular drug”, as used herein, refers to a very large molecule (preferably >900 Daltons, 1 k-5 k, 5 k-10 k, 10 k-50 k, 50 k-100 k Daltons), such as protein, commonly created by polymerization of smaller subunits (monomers) that provide therapeutic effects upon administration to cells or subjects. The most common examples of macromolecular drugs biopolymers (nucleic acids, proteins, carbohydrates and polyphenols) and large non-polymeric molecules (such as lipids and macrocycles).

“Chromophore”, as used herein, is a molecule that absorbs certain wavelengths of visible light and transmits or reflects others resulting in the appearance of color.

“Fluorophore”, as used herein, is a fluorescent chemical compound that can re-emit light upon light excitation. Fluorophores typically contain several combined aromatic groups, or plane or cyclic molecules with several π bonds.

“Subject”, as used herein, refers to an animal, preferably a mammal, more preferably a human.

The term “Interaction” or “interacts”, as used herein, refers to the physical relationship between an active pharmaceutical ingredient and a synthetic stem, for example, via attachment, adherence, or binding.

The term “nucleic acid” refers to single-stranded or double-stranded DNA or RNA; preferably the nucleic acid is 10 kb or less (5 kb, 2 kb, 1 kb, 500 bp) in length and may be coding or non-coding. An “oligonucleotide” is a short nucleic acid, may include PNA, RNA or DNA or both, and may be 8-500 nucleotides or base pairs, preferably, 10-250, 15-30, 15-50, and 20-300.

“Antibody”, as used herein, covers monoclonal antibodies, polyclonal antibodies, dimers, multimers, multispecific antibodies (eg. bispecific antibodies), veneered antibodies, antibody fragments and small immune proteins (SIPs) (see Int. J. Cancer (2002) 102, 75-85). An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, ie. a molecule that contains an antigen binding site that immunospecifically binds an antigen of a target of interest or part thereof. The antibodies may be of any type—such as IgG, IgE, IgM, IgD, and IgA)—any class—such as IgG1, IgG2, IgG3, IgG4, IgA1 and IgA2—or subclass thereof. The antibody may be or may be derived from murine, human, rabbit or from other species.

“Antibody fragments”, as used herein, refers to a portion of a full length antibody, generally the antigen binding or variable region thereof. Examples of antibody fragments include, but are not limited to, Fab, Fab′, F(ab′)2, and Fv fragments; diabodies; linear antibodies; single domain antibodies, including dAbs, camelid VHH antibodies and the IgNAR antibodies of cartilaginous fish. Antibodies and their fragments may be replaced by binding molecules based on alternative non-immunoglobulin scaffolds, peptide aptamers, nucleic acid aptamers, structured polypeptides comprising polypeptide loops subtended on a non-peptide backbone, natural receptors or domains thereof.

The present invention relates to an aqueous dispersion of chemical compositions useful in preparations of compositions comprising active pharmaceutical ingredients.

The invention relates to liquid compositions comprising heterocyclic ring systems that interact with proteins through non-covalent interactions. The non-covalent interactions between heterocyclic rings and protein molecules comprise interactions ranging from electrostatic interactions, hydrogen bond interactions, van der Waals interactions, and hydrophobic interactions. While not being bound to any theory, it is believed that these interactions may occur through electrostatic or hydrophobic (including pi-pi interactions) forces. In some embodiments the active pharmaceutical ingredient is covalently linked to the stem which is capable of undergoing hydrolysis or bond breakage leading to the release of pharmaceutical ingredient under physiological conditions.

The present invention also relates to methods of enhancing a biological response to an active pharmaceutical ingredient via composition of the active pharmaceutical ingredient with the heterocyclic ring systems.

The present invention also relates to methods for preparation and use of these compositions of active pharmaceutical ingredient with the heterocyclic ring systems either prophylactically and/or therapeutically.

In one exemplary embodiment, the chemical compositions comprise a hydrophobic organic material stable to aqueous hydrolysis. Examples of hydrophobic organic materials useful in the present invention include, but are in no way limited to, organic waxes such as bees wax and carnauba wax, cetyl alcohol, ceteryl alcohol, behenyl alcohol, fatty acids, and fatty acid esters. Preferred for use in chemical compositions is an organic wax with a melting point above 25° C. In some embodiments, the hydrophobic organic materials may further comprise pharmaceutically acceptable oil. Examples of pharmaceutically acceptable oils include, but are not limited to, mineral oil, oils of vegetable origin (maize, olive, peanut, soybean etc.) and silicone fluids such as Dow Corning DC200. Some of these embodiments may comprise 1% to 100% of an organic wax with a melting point above 25° C. and 0 to 99% of pharmaceutically acceptable oil.

In one exemplary embodiment, the chemical compositions comprise a stabilizing component. Examples of such stabilizing components include, but are not limited to chitosan, charged emulsifiers such as sodium dodecyl sulfate and fatty acids or salts thereof. Examples of fatty acids include, but are not limited to, myristic acid and behenic acid.

In one exemplary embodiment, the chemical compositions comprise an emulsifying component. Examples of such emulsifying components include emulsifiers but are not limited to cetyl trimethylammonium bromide and cetyl pyridinium halide, chitosan, sodium dodecyl sulfate, N-[1-(2,3-Dioleoyloxy)]-N,N,N-trimethylammonium propane methylsulfate (DOTAP), sodium myristate, Tween 20, Tween 80 (polyoxyethylene sorbitan monoloaurate), polyethylene stearyl ether, Dioctyl sodium sulfosuccinate such as AOT, Brij700 and combinations thereof and. As is understood by the skilled artisan upon reading this disclosure, alternative emulsifiers can also be used. The emulsification component may be present in a level from about 0.01% to about 10% or from about 0.05% to about 5% or from about 0.1% to about 2% or from about 0.5% 5 to about 2% or from about 1.0% to about 2.0%.

In some embodiments the chemical compositions further comprise moieties that are ligands for surface receptors on the cells where the pharmaceutical ingredients are to be delivered, and target the pharmaceutical ingredients to those cells. For example, a polysaccharide recognized by cell surface receptors such as mannose receptor can be linked to the synthetic stem of the chemical composition, thereby improving delivery of the synthetic stem comprising pharmaceutical ingredients into the cells carrying those receptors.

In some embodiments, the chemical composition of the present invention is prepared via a process essentially free from organic solvents.

In one embodiment, the chemical compositions are subjected to curing process in presence of molten lipid or wax. As a non-limiting example, in one embodiment the solid lipid or wax above its melt temperature to form a molten lipid or wax. The molten material is then dispersed into the chemical composition comprising a synthetic stem, heterocyclic rings and pharmaceutical ingredients using an ultrasonic horn, or a high-pressure homogenizer. The resultant emulsion comprising chemical composition and molten materials is then allowed to cool down to room temperature.

In one embodiment of the present invention, the chemical compositions are useful in delivery of vaccines, wherein the active pharmaceutical ingredient is a protein, preferably a subunit vaccine antigen such as, but not limited to, tetanus toxoid or gp140, or a nucleic acid such as, but not limited to, DNA, RNA, siRNA, ShRNA, or an antisense oligonucleotide. These embodiments may further comprise an anionic adjuvant such as, but not limited to, poly(IC) or CpGB.

In one embodiment, wherein the active pharmaceutical ingredient in a subunit vaccine antigen and the chemical composition is a vaccine formulation, the composition can be administered to a subject to immunize the subject against an antigen.

Active pharmaceutical ingredients used in the chemical compositions include, but are in no way limited to, drugs, including vaccines, nutritional agents, cosmeceuticals and diagnostic agents. Examples of active pharmaceutical ingredients for use in the present invention include, but are not limited to analgesics, anti-anginal agents, anti-asthmatics, anti-arrhythmic agents, anti-angiogenic agents, antibacterial agents, anti-benign prostate hypertrophy agents, anti-cystic fibrosis agents, anti-coagulants, anti-depressants, anti-diabetic agents, anti-epileptic agents, anti-fungal agents, antigout agents, anti-hypertensive agents, anti-inflammatory agents, anti-malarial agents, anti-migraine agents, anti-muscarinic agents, anti-neoplastic agents, anti-obesity agents, anti-osteoporosis agents, anti-parkinsonian agents, anti-protozoal agents, anti-thyroid agents, anti-urinary incontinence agents, anti-viral agents, anxiolytics, beta-blockers, cardiac inotropic agents, cognition enhancers, corticosteroids, COX-2 inhibitors, diuretics, erectile dysfunction improvement agents, essential fatty acids, gastrointestinal agents, histamine receptor antagonists, hormones, immunosuppressants, keratolyptics, leukotriene antagonists, lipid regulating agents, macrolides, muscle relaxants, non-essential fatty acids, nutritional agents, nutritional oils, protease inhibitors and stimulants.

Chemical compositions of the present invention are thus useful prophylactically and therapeutically in treatment of a subject suffering from a disorder or disease treatable with the active pharmaceutical ingredient present in the composition.

The chemical compositions of the present invention are useful in methods of targeting an active pharmaceutical ingredient to a selected cell or tissue and producing pharmaceutical formulations targeted to a selected cell or tissue. In these methods, a chemical composition comprises a heterocyclic ring component which binds to the protein through non-covalent interactions. The chemical formulations further comprise the active pharmaceutical ingredient to be targeted to the cell or to tissue that produces therapeutic benefits upon release from the chemical composition.

The embodiments of the invention described above are intended to be merely exemplary; numerous variations and modifications will be apparent to those skilled in the art. All such variations and modifications are intended to be within the scope of the present invention as defined in any appended claims.

The following nonlimiting examples are provided to further illustrate the present invention.

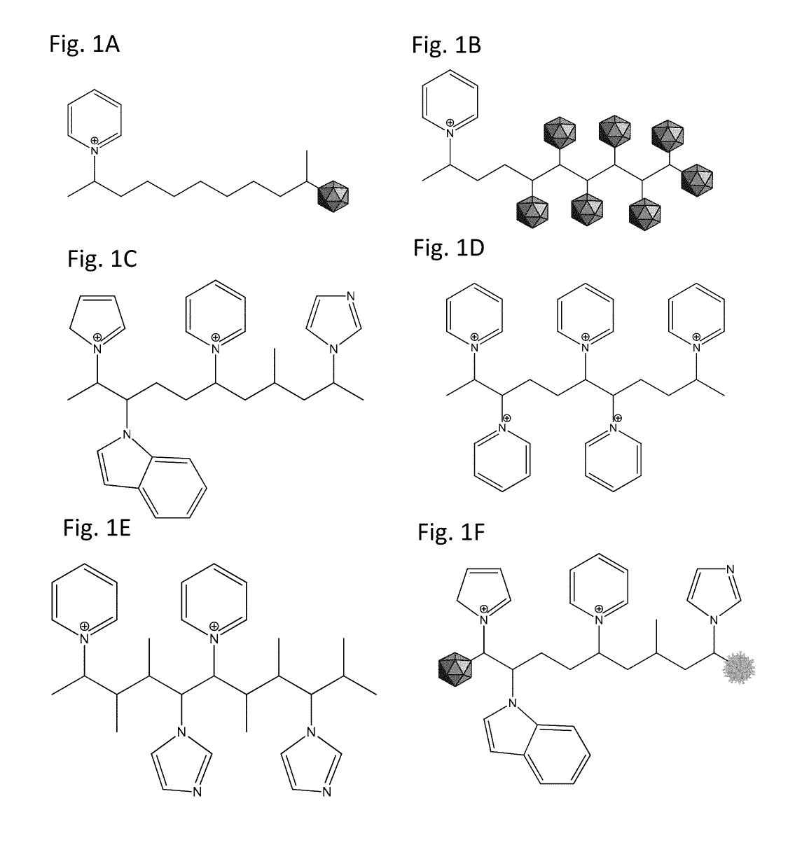

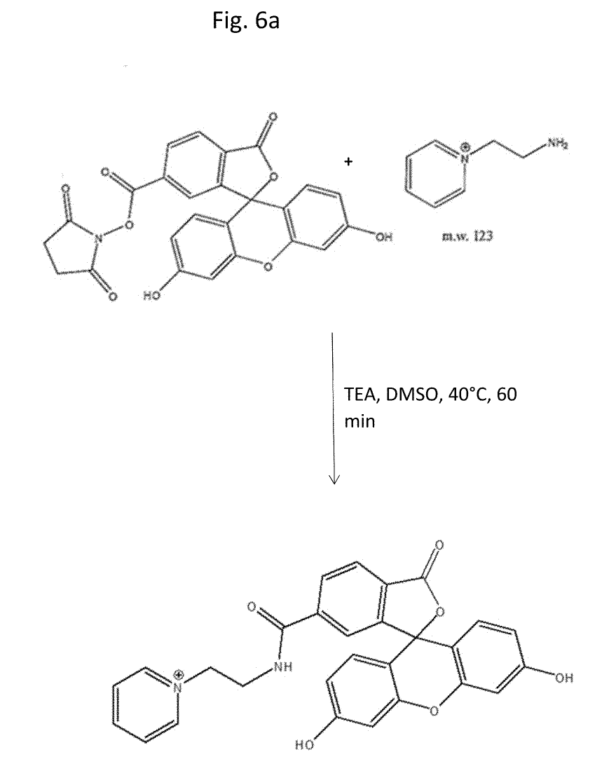

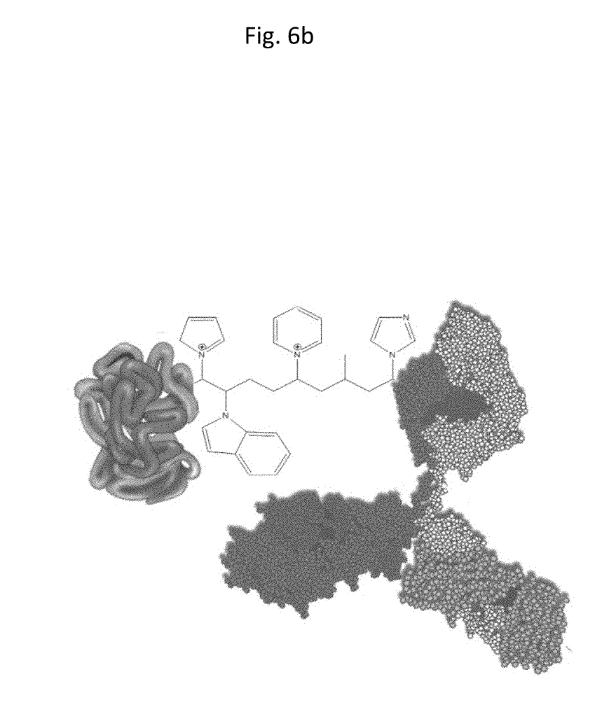

In some embodiments described below pyridinium ring systems have been used as a model example to illustrate the usage in various applications. This invention should in no way be construed as being limited only to pyridinium ring compounds. As exemplified in FIGS. 1A-1F, 6A-F, 8 and 9, other heterocyclic systems, for instance, pyrolidine ring systems, piperdine ring systems, oxalane ring systems, indole ring systems, thian ring systems, oxepine ring systems etc can be used in place of pyridine ring systems. One of skill would understand that based on the teachings described in the specification and armed with common knowledge in art, one can readily substitute pyridinium ring systems with other heterocyclic ring systems described in the specification to achieve similar embodiments that possess similar utility as seen in case of pyridinium based embodiments. The invention also contemplates several embodiments wherein multiple species of heterocyclic ring systems are present and also multiple copies of the same heterocyclic ring systems being present. These embodiments shall be used to generate compounds with similar utility as described in the examples.

### Example 1—Synthesis of Pyridinium Constructs

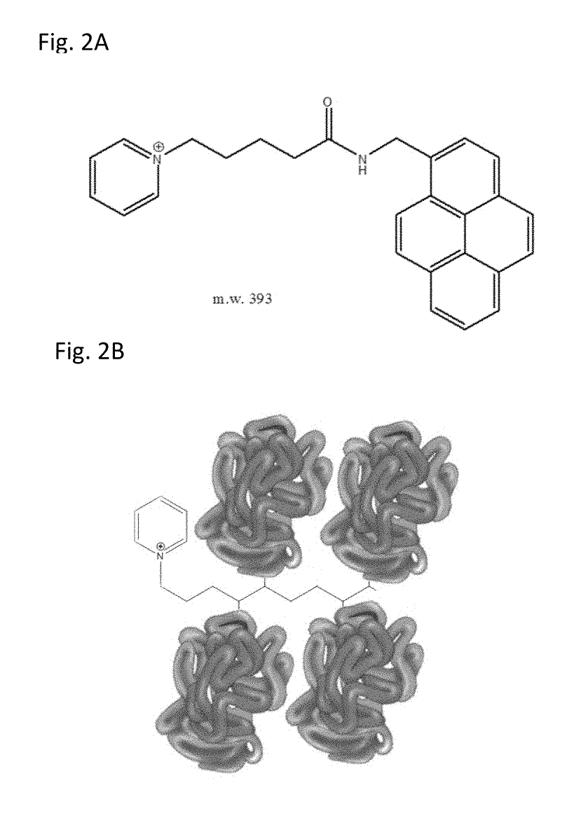

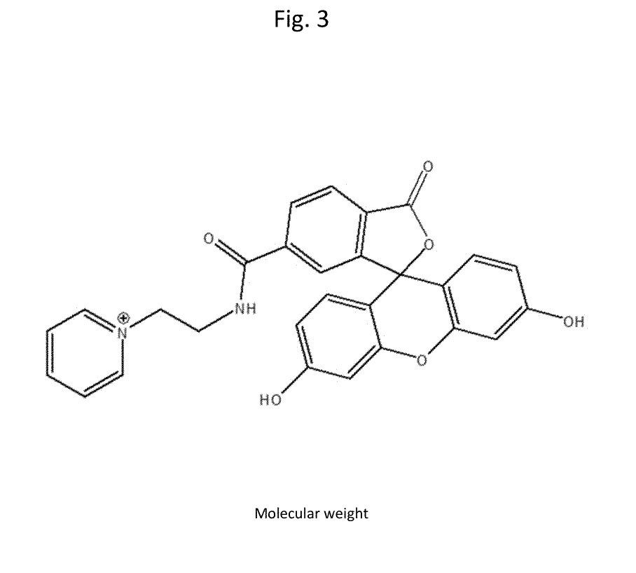

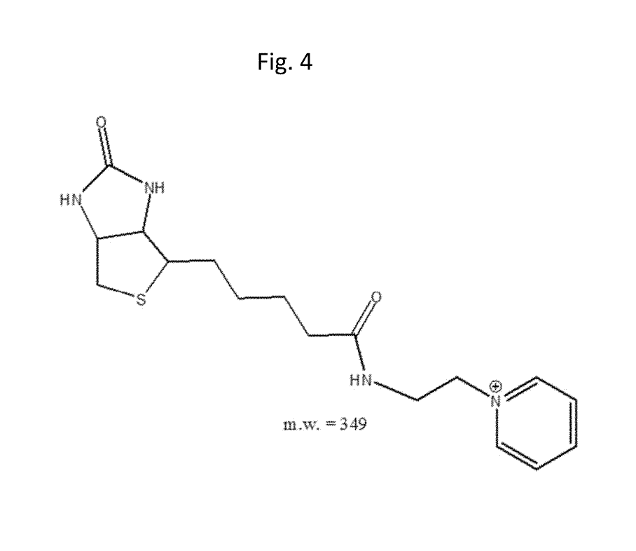

FIGS. 1A-1F show non-limiting examples of heterocyclic chemical compositions wherein more than one kind of heterocyclic ring system is attached to the synthetic stem. It also shows heterocyclic chemical compositions that further comprise a pharmaceutical ingredient (represented as icosahedrons) or a diagnostic marker (represented as sphere with dots) or combinations thereof. FIGS. 2A-2B, 3 and 4 show non limiting examples of constructs comprising pyridinium ring systems with chromophore, fluorophore or biotin.

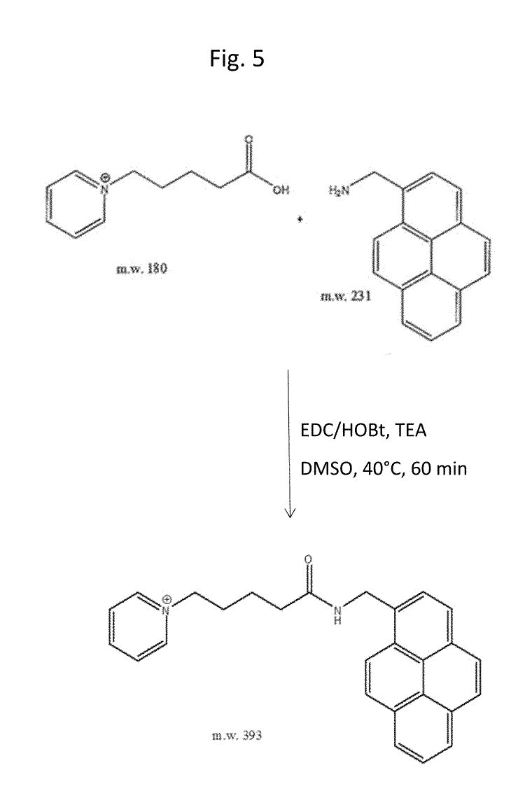

The following example details the synthetic process utilized in the production of chemical composition comprising a heterocyclic pyridinium ring, synthetic stem and chromophore as shown in FIG. 5. One of skill in the art would understand that this synthetic process can be optionally modified using knowledge in art to produce chemical formulations comprising multiple heterocyclic rings and multiple chromophores The synthetic coupling reactions for the pyridinium constructs used peptide coupling reactions, whereby a primary amine and a carboxylic acid react together to form an amide bond, using EDC (1-Ethyl-3-(3-dimethylaminopropyl)-carbodiimide) as a zero-length crosslinking agent. Specifically, 1-(4-carboxybutyl) pyridinium (100 mg; 0.55 mmol), pyrenemethylamine (127 mg; 0.55 mmol), EDC (150 mg; 0.78 mmol) and HOBt (140 mg; 100 mmol) were dissolved in 2 mL of DMSO. A drop of triethylamine was added. The reaction was heated to 35° C. for 30 minutes and injected crude onto a 26 g C18 column for purification (mobile phase A: 10 mmol ammonium formate, mobile phase B: acetonitrile, gradient: 5-95% aqueous), to recover the final product 1-(5-oxo-5-(pyren-1-ylmethylamino)pentyl)pyridinium (m.w.=393). 1H NMR (predicted, δ) 1.3 (4H, m), 1.53 (2H, m), 2.13 (2H, t), 4.91 (2H, s), 7.62 (1H, m), 7.71 (4H, m), 7.82 (1H, m), 7.88 (1H, m), 8.00 (1H, m), 8.12 (1H, m), 8.18 (1H, d), 8.22 (2H, m), 8.74 (1H, m), 8.89 (2H, m).

The following example details the synthetic process utilized in the production of chemical composition comprising a heterocyclic pyridinium ring, synthetic stem and fluorophore as shown in FIG. 6. One of skill in the art would understand that this synthetic process can be optionally modified using knowledge in art to produce chemical formulations comprising multiple heterocyclic rings and multiple fluorophores.

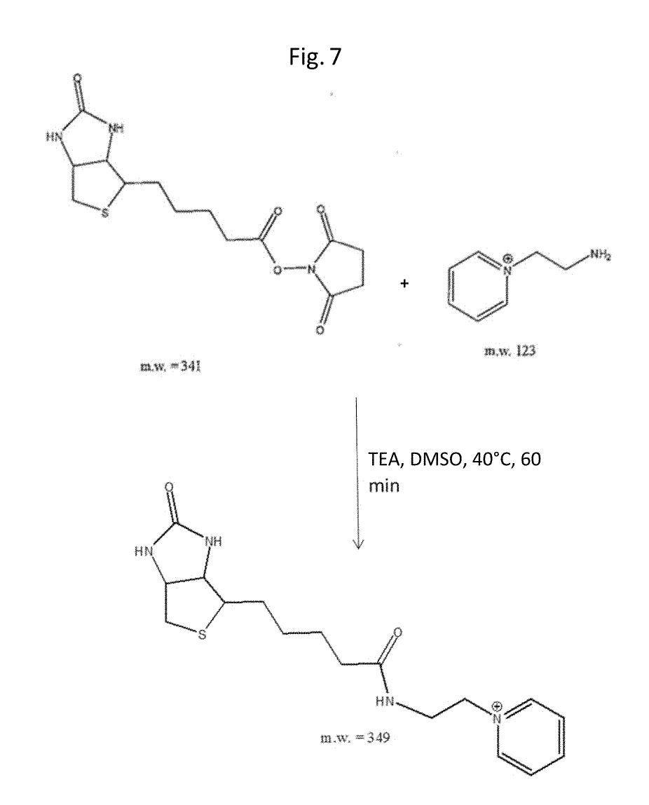

The synthetic coupling reactions for the pyridinium constructs used peptide coupling reactions, whereby a primary amine and a NHS-ester react together to form an amide bond, using EDC (1-Ethyl-3-(3-dimethylaminopropyl)-carbodiimide) as a zero-length crosslinking agent. Specifically, NHS-fluorescein (100 mg; 0.211 mmol), 1-(2-aminoethylpyridinium) (37 mg; 0.3 mmol), and EDC (58 mg; 0.3 mmol) were dissolved in 2 mL of DMSO. A drop of triethylamine was added. The reaction was heated to 35 C for 30 minutes and injected crude onto a 26 g C18 column for purification (mobile phase A: 10 mmol ammonium formate, mobile phase B: acetonitrile, gradient: 5-95% aqueous), to recover the final product 1-(2-(3′,6′-dihydroxy-3-oxo-3H-spiro[isobenzofuran-1,9′-xanthene]-6-ylcarboxamido) ethyl)pyridinium (m.w. 481). 1H NMR (predicted, δ) 1.60 (2H, t), 3.02 (2H, t), 6.40 (2H, m), 6.62 (2H, m), 7.15 (2H, m), 7.98 (1H, s), 8.07 (1H, m), 8.16 (1H, m), 8.22 (2H, m), 8.56 (1H, s), 8.74 (1H, t), 8.89 (2H, m), 9.89 (2H, s) The following example details the synthetic process utilized in the production of chemical composition comprising a heterocyclic pyridinium ring, synthetic stem and biotin as shown in FIG. 7. One of skill in the art would understand that this synthetic process can be optionally modified using knowledge in art to produce chemical formulations comprising multiple heterocyclic rings and multiple units of biotin

The synthetic coupling reactions for the pyridinium-biotin constructs used peptide coupling reactions, whereby a primary amine and a NHS-ester react together to form an amide bond, using EDC (1-Ethyl-3-(3-dimethylaminopropyl)-carbodiimide) as a zero-length crosslinking agent. Specifically, NHS-biotin (68 mg; 0.2 mmol), 1-(2-aminoethylpyridinium) (37 mg; 0.3 mmol), and EDC (58 mg; 0.3 mmol) were dissolved in 2 mL of DMSO. A drop of triethylamine was added. The reaction was heated to 35° C. for 30 minutes and injected crude onto a 26 g C18 column for purification (mobile phase A: 10 mmol ammonium formate, mobile phase B: acetonitrile, gradient: 5-95% aqueous) recovering final product 1-(2-(5-(2-oxohexahydro-1H-thieno[3,4-d]imidazol-4-yl)pentanamido)ethyl)pyridinium (m.w. 349). 1H NMR (predicted, δ) 1.25 (2H, m), 1.62 (6H, m), 2.13 (2H, t), 2.85 (2H, d), 3.15 (2H, t), 3.36 (1H, m), 4.59 (2H, m), 6.0 (2H, s), 8.01 (1H, s), 8.22 (2H, m), 8.74 (1H, m), 8.89 (2H, m).

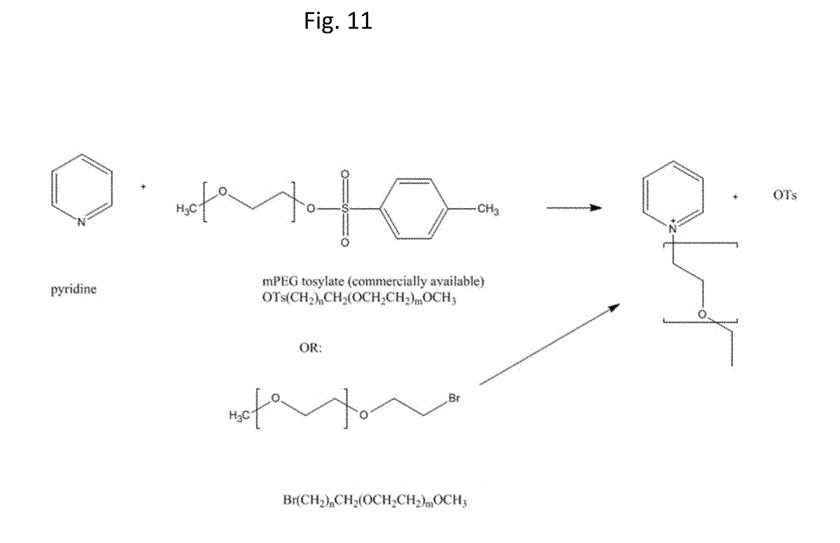

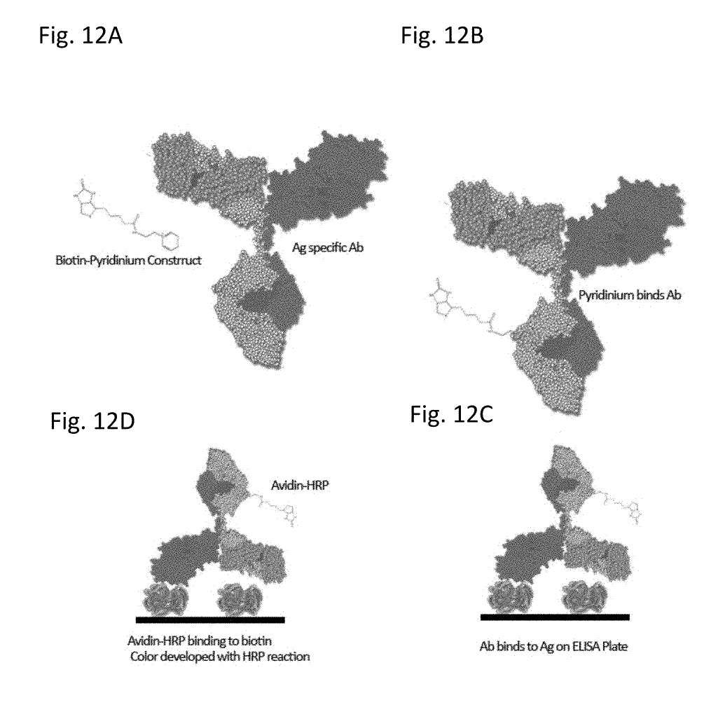

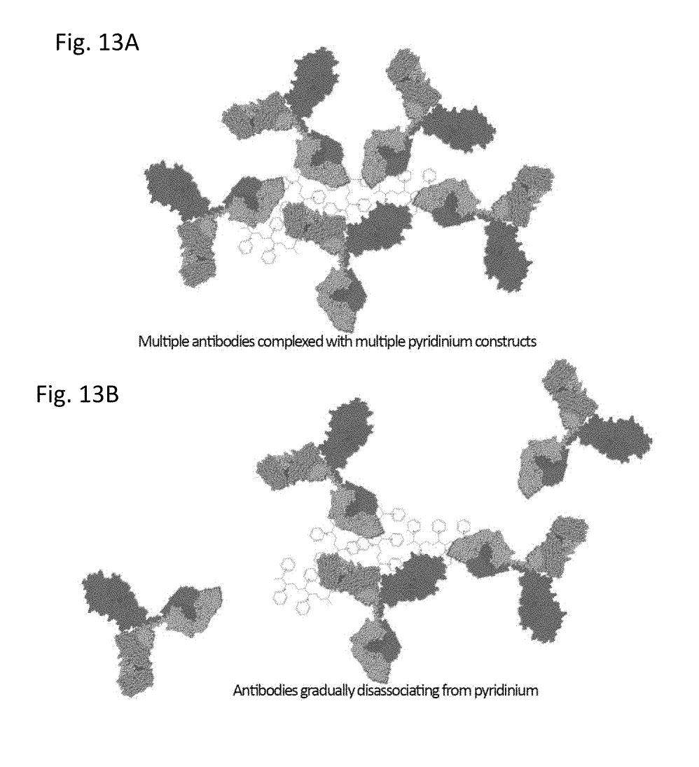

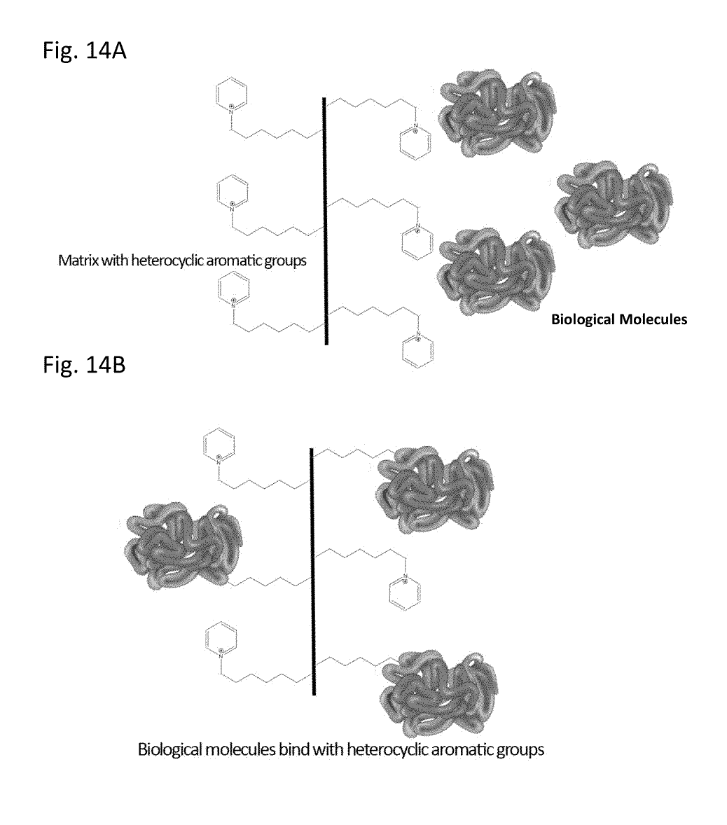

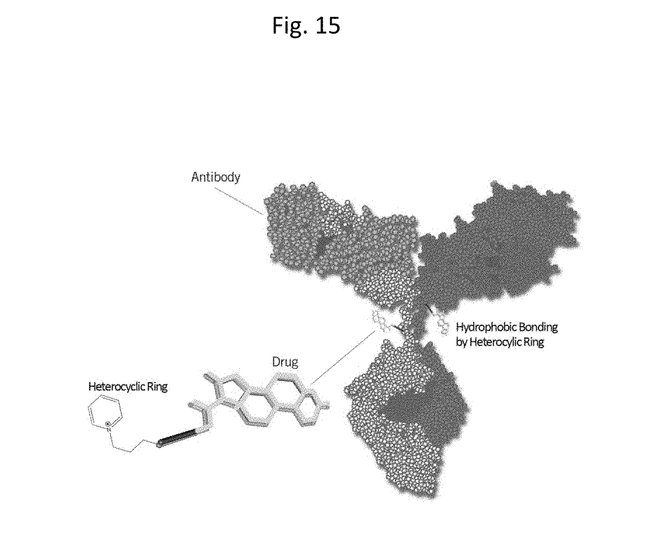

One of skill in the art would also readily appreciate that the synthetic schemes described above are not limited to pyridinium based heterocyclic ring systems but rather shall be modified to suit other heterocyclic ring systems described in FIGS. 8 and 9. The synthetic process can be readily modified with knowledge in art to generate several versions of constructs, a few of which are exemplified in FIGS. 1A-1F. FIG. 11 is a non-limiting example that shows the synthesis of PEGylated pyridinium constructs.

### Example 2—Quantitative Analysis of Binding of Pyridinium Constructs with Biological Molecules Using Fluorescence Assay

This example illustrates the interactions between the pyridinium constructs containing fluorophores and proteins. Human IgG1 or albumin shall be used as the biomolecule of interest. The pyridinium constructs containing fluorophore is synthesized and purified as described in Example 1.

The pyridinium construct containing fluorophore is mixed with human IgG1 or albumin, in solution. Unbound, free pyridinium construct can be separated from the protein and protein-bound-construct by molecular sieve filtration; for example, using 10,000 mw cut off membrane the unbound pyridinium construct will readily pass through the membrane but proteins like the 150,000 mw IgG1 is retained. The protein containing fraction can then be assayed for retained fluorescence. The retained fluorescence activity in the protein fraction represents the pyridinium-constructs' binding to the proteins.

Alternatively, the unbound fluorescent-pyridinium construct coming through the filter can also be measured. When a fluorescent-pyridinium construct binds proteins, the amount of fluorescence coming through the filter decreases as the amount of protein included in the initial binding reaction increases. Assays will use fluorescent-pyridinium construct incubated alone and, for comparison, with increasing amounts of protein. The subsequent analysis evaluates the amount of unbound fluorescent-pyridinium-construct coming through the membranes. All fluorescence measurements are carried out in a Fluorimeter following standard protocols established in art.

Alternatively, the fluorescence binding assay shall also be conducted by keeping the amount of the protein constant and titrating in the fluorescent-pyridinium construct. Fluorophore not covalently linked to pyridinium is titrated in separate experiment along with protein and is used as background fluorescence measurement for the concentration range. After incubation the protein is separated from unbound construct or unbound fluorophore and the fluorescence of the protein fraction is measure. The difference in the fluorescence between these two binding assays (construct vs. fluorophore alone) semi-quantitatively represents the binding resulting from pyridinium binding to with to protein.

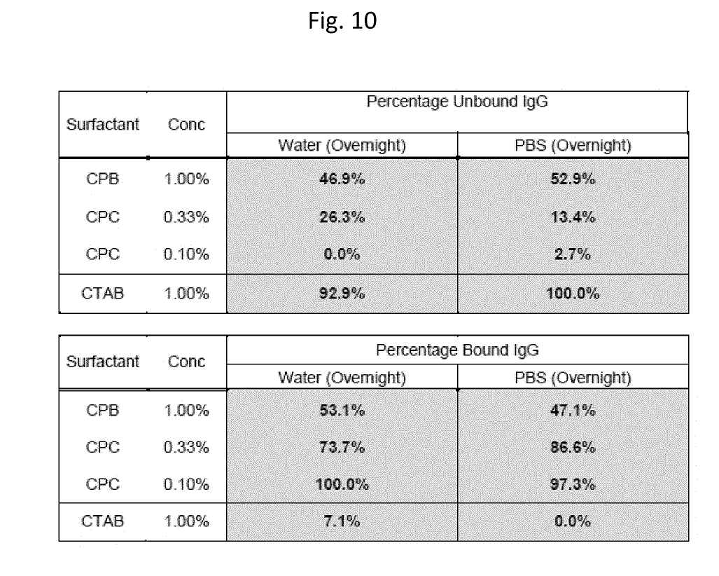

The data table shown in FIG. 10 indicates the results from an binding experiments between a protein (IgG) and pyridinium constructs such as CPC (cetyl pyridinium chloride), and CPB (Cetyl pyridinium bromide). For comparison, a non-heterocyclic molecule such as CTAB (Cetyl trimethylammonium bromide) was also tested for binding with IgG under PBS and water conditions.

The CPC (cetyl pyridinium chloride) was tested at 0.33 wt/vol. % and 0.10 wt/vol % and CPB was tested at lwt/vol %. In separate trials, the experiment was done in presence of water and PBS buffer solutions. The percentage of unbound IgG and pyridinium bound IgG were determined by measuring the free IgG (protein) after the protein was allowed to bind overnight with pyridinium constructs. The results indicate that CPC and CPB bind to IgG well even in PBS buffer, which contains the anti-chaotropic agent phosphate. The strong binding of CPC and CPB to protein even in the presence of antichaotropic agents indicate that the binding between the protein and the pyridinium constructs is hydrophobic in nature. The results also indicate that lower concentrations of CPC and CPB based constructs resulted in better binding of proteins in both water and PBS. CTAB on the other hand, lacks heterocyclic ring systems but has the same charge as CPC or CPB, nevertheless it did not bind to the IgG protein under the conditions indicating that the CTAB is unable to bind to IgG through hydrophobic or electrostatic interactions. Similar assays can be carried out to identify the optimal concentration of pyridinium constructs that allow better binding to other proteins of interest.

The nature of interactions that occur between pyridinium constructs and proteins can be ascertained by using the following experiment. The binding assay described above shall be repeated in the presence of anti-chaotropic agents/salts like phosphate, which disrupt electrostatic interactions and strengthen hydrophobic interactions. If the binding interactions between pyridinium constructs and proteins remain unaffected, or possibly strengthened, by the addition of (antichaotropic salts) phosphate during incubation, then it implies that the interaction between the pyridinium constructs and proteins is hydrophobic in nature. Conversely one may also add a chaotropic agent like ethanol to binding reactions which should negatively affect hydrophobic binding, but not electrostatic binding. If the binding interactions between pyridinium constructs and proteins get reduced, or possibly weakened, by the addition of (chaotropic reagents) ethanol during incubation, then it would also imply that the interaction between the pyridinium constructs and proteins is hydrophobic in nature.

Another variation of quantitative measurement is to measure the fluorescence exhibited by free verses bound fluorescent-pyridinium constructs in equilibrium reactions where the amount of fluorescent pyridinium-construct is held constant and proteins are titrated into the binding reactions. The protein in these reactions is separated from free construct by molecular seize membrane and the amount of free fluorescence is measured; the amount of pyridinium construct that is free verses bound in then calculated. From these analyses the binding constants and affinities of these interactions can be calculated as described by Pollard et al. (Thomas Pollard, in Mol. Biol of the Cell, Vol. 21: 4061-67, 2010). Analyses of these binding constants in the presence of anti-chaotropic and chaotropic agents would indicate that pyridinium is binding to the proteins by hydrophobic interactions.

Another variation of quantitative measurement is to use Microscale Thermophoresis to measure fluorescence of pyridinium constructs bound to proteins. (nanotemper-technologies.com). MicroScale Thermophoresis is the directed movement of microscopic entities or biopolymers or macromolecules in a microscopic temperature gradient. Any change of the hydration shell of biomolecules due to changes in their structure/conformation results in a relative change of the movement along the temperature gradient and is used to determine binding affinities. MST allows measurement of interactions directly in solution without the need of immobilization to a surface (immobilization-free technology). MST can efficiently measure the dissociation constant (Kd) of fluorescent ligand interacting with protein, or other large biological molecules.

MST is based on the directed movement of molecules along temperature gradients, an effect termed thermophoresis. A spatial temperature difference ΔT leads to a depletion of molecule concentration in the region of elevated temperature, quantified by the Soret coefficient S_{T}: cₕₒₜ/c_{cold}=exp(−S_{T}ΔT)

Thermophoresis depends on the interface between molecule and solvent. Under constant buffer conditions, thermophoresis probes the size, charge and solvation entropy of the molecules. The thermophoresis of a fluorescently labeled molecule (A) typically differs significantly from the thermophoresis of a molecule-target complex (AT) due to size, charge and solvation entropy differences. This difference in the molecule's thermophoresis is used to quantify the binding in titration experiments under constant buffer conditions.

The thermophoretic movement of the fluorescently labeled molecule is measured by monitoring the fluorescence distribution (F) inside a capillary. The microscopic temperature gradient is generated by an IR-Laser, which is focused into the capillary and is strongly absorbed by water. The temperature of the aqueous solution in the laser spot is raised by up to ΔT=5 K. Before the IR-Laser is switched on a homogeneous fluorescence distribution F_{cold} is observed inside the capillary. When the IR-Laser is switched on, two effects, separated by their time-scales, contribute to the new fluorescence distribution Fₕₒₜ. The thermal relaxation time is fast and induces a binding-dependent drop in the fluorescence of the dye due to its local environmental-dependent response to the temperature jump. On the slower diffusive time scale (10 s), the molecules move from the locally heated region to the outer cold regions. The local concentration of molecules decreases in the heated region until it reaches a steady-state distribution.

While the mass diffusion (D) dictates the kinetics of depletion, S_{T} determines the steady-state concentration ratio cₕₒₜ/c_{cold}=exp(−S_{T} ΔT)≈1−S_{T} ΔT under a temperature increase ΔT. The normalized fluorescence Fₙₒᵣₘ=Fₕₒₜ/F_{cold} measures mainly this concentration ratio, in addition to the temperature jump ∂F/∂T. In the linear approximation: Fₙₒᵣₘ=1+(∂F/∂T−S_{T})ΔT. Due to the linearity of the fluorescence intensity and the thermophoretic depletion, the normalized fluorescence from the unbound molecule Fₙₒᵣₘ(A) and the bound complex Fₙₒᵣₘ(ΔT) superpose linearly. By denoting x the fraction of molecules bound to targets, the changing fluorescence signal during the titration of target T is given by: Fₙₒᵣₘ=(1−x) Fₙₒᵣₘ(A)+x Fₙₒᵣₘ(ΔT).

Quantitative binding parameters shall be obtained by using a serial dilution of the binding substrate. By plotting Fₙₒᵣₘ against the logarithm of the different concentrations of the dilution series, a sigmoidal binding curve is obtained. This binding curve can directly be fitted with the nonlinear solution of the law of mass action, to determine dissociation constant K_{D}. Similarly dissociation constant shall be determined in presence of chaotropic and antichaotrophic reagents as described earlier.

### Example 3—Quantitative Analysis of Binding of Pyridinium Constructs with Biological Molecules Using Biotin Assay

This example illustrates the interactions between the pyridinium constructs containing biotin and proteins. Human IgG1 or albumin shall be used as the biomolecule of interest. The pyridinium construct containing biotin is synthesized and purified as described in Example 1.

Surface Plasmon Resonance (SPR) can be used to quantitate the binding of pyridinium constructs containing biotin to proteins. Surface plasmon resonance (SPR) is the resonant oscillation of conduction electrons at the interface between a negative and positive permittivity material stimulated by incident light. The resonance condition is established when the frequency of incident photons matches the natural frequency of surface electrons oscillating against the restoring force of positive nuclei. SPR in subwavelength scale nanostructures can be polaritonic or plasmonic in nature.

SPR is the basis of many standard tools for measuring adsorption of material onto planar metal (typically gold or silver) surfaces or onto a metallic surface. It is the fundamental principle behind many color-based biosensor applications and different lab-on-a-chip sensors. Biacore instruments shall be used to perform SPR measurements unless indicated otherwise. (https://www.biacore.com/lifesciences/index.html)

Biacore can measure mass accumulation as proteins bind to ligands that are immobilized on the surface of the sensor chip. In this example, avidin coated sensor chip would be used for SPR measurements. First, the avidin coated sensor chips are treated with pyridinium-biotin constructs to ensure that avidin coated chip surface is saturated with pyridinium-biotin constructs. This is possible due to the high affinity between biotin and avidin (KD of approximately 10-15 M).

Through a microflow system, a solution with the protein of interest is injected over the pyridinium construct covered sensor chip surface. As the protein binds the pyridinium constructs, an increase in SPR signal (expressed in response units, RU) is observed. After desired association time, a solution without the protein (usually buffer containing antichaotropic agents) is injected on the microfluidics that will enable the dissociation of the bound complex between pyridinium construct and protein. The dissociation of complex between the pyridinium construct and protein ligand will result in a decrease in SPR signal (expressed in resonance units, RU). From these association (‘on rate’, ka) and dissociation rates (‘off rate’, kd), the equilibrium dissociation constant (‘binding constant’, KD) can be calculated. (K_{D}=k_{d}/kₐ).

### Example 4—Pyridinium-Biotin Constructs in ELISA Semi-Quantitative Analyses

Enzyme linked immunosorbent assay (ELISA) is an analytic biochemistry assay that uses a solid-phase enzyme immunoassay (EIA) to detect the presence of a substance, usually an antigen, in a liquid sample. ELISA screening assays can be used to determine the affinity of binding of a particular pyridinium construct to a variety of proteins.

First the multiwell plate commonly used in ELISA is coated with proteins of interest, the antigen. Separately antibody proteins with specificity for desired antigen are incubated with the pyridinium-biotin construct, allowing the pyridinium to non-covalently bind to the antibody protein which links (labels) the biotin to the antibody This biotin labeled antibody is then added to the wells at various concentrations and incubated, allowing the antibody to bind specifically to the antigen. After this incubation and a wash, the plates are subsequently incubated with avidin-horseradish peroxidase conjugate or avidin-alkaline phosphatase conjugate, which are standard ELISA reagents. The avidin will bind to the biotin that is linked to the antibody bound specifically to the antigen. After subsequently washing away unbound avidin-conjugate, the ELISA colorimetric reactions are developed using standard ELISA substrates for horseradish peroxidase or alkaline phosphatase and read on an ELISA plate reader.

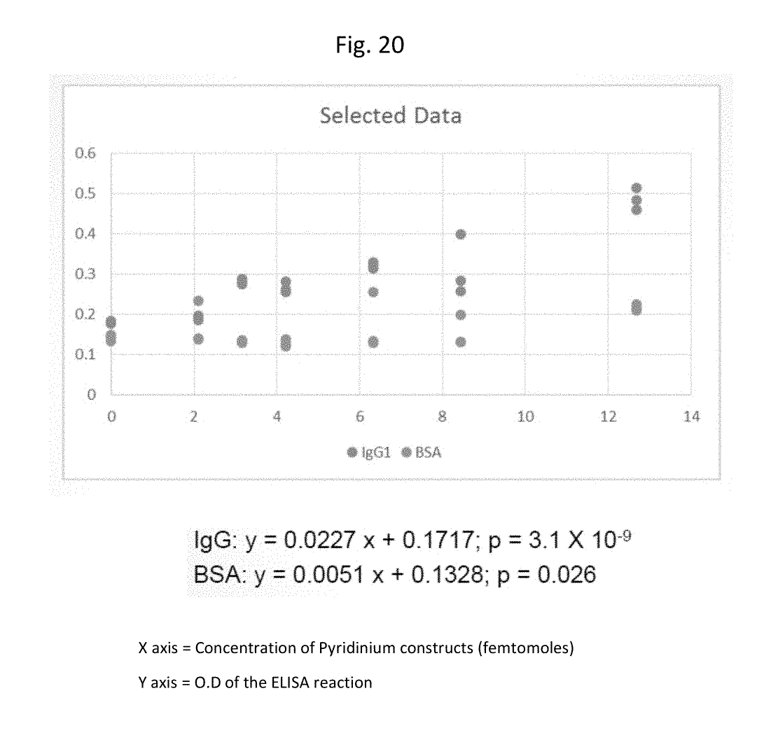

Another variation of ELISA assay is one in which the plate wells are first coated with rabbit IgG. After a wash with PBS buffer, the IgG coated wells are blocked with bovine serum albumin (BSA) to saturate the remaining surface of the wells in order to lower background noise. A set of control wells are coated and blocked with BSA only as described above. The IgG coated and BSA blocked wells are then reacted with pyridinium-biotin construct in PBS buffer. Increasing amounts of with pyridinium-biotin construct in femtomole range are added in different wells. Those wells are washed with PBS buffer and reacted with constant amount of avidin-Horseradish peroxidase (HRP) in PBS. The colorimetric product of the reaction is then detected by an ELISA plate reader. The results of the ELISA experiment are shown in FIG. 20.

The difference in dilution curves at lower concentration of IgG vs. BSA for pyridinium constructs are quite different indicating the strong binding affinity of IgG even at lower concentrations when compared with that of BSA in presence of PBS buffer.

The ELISA experiment may be run in presence of varying concentration of antichaotropic agents to determine which protein exhibits the strongest hydrophobic interaction with a particular pyridinium construct. Likewise the same assay may be repeated with varying concentrations of chaotropic agents to identify the protein-pyridinium construct pair that is resilient to external perturbations such as pH or charges.

### Example 5—Pyridinium-Fluorochrome Constructs to Label Antigen Specific Antibodies for the Detection and Semi-Quantitation of Antigens

Fluorochrome-pyridinium constructs can be used to fluorescently label biological molecules, including antibodies, which can specifically bind to its receptor, ligand, or antigen (in the example of antibodies). This binding between biological molecule labeled by the pyridinium-fluorochrome and its ligand is detected by a fluorometer or fluorescence microscope.

When the ligand is expressed on the surface of cells or in tissues, the binding between the biological molecule and cells or tissues are detected and semi-quantitated using fluorescence microscopy.

When the ligand is purified molecule, it is adsorbed to wells in assay plate, like those used for ELISA, and then the biological molecule labeled by the pyridinium-fluorochrome can be incubated in the wells coated with the ligand. After washing the well removing unbound materials, the remaining bound fluorescently labeled biological molecules are measured by a fluorometer.

### Example 6—Use of Pyridinium Constructs Having Multiple Copies of Pyridinium to Generate a Sustained Release Formulation of Biological Molecules

Pyridinium constructs like that in FIG. 1D and larger synthetic stems with larger numbers of covalently bound pyridinium are incubated with therapeutic biological molecules, including antibody proteins with a therapeutic antigen specificity. Multiple copies of the therapeutic biological molecule are hydrophobically, non-covalently bound to the pyridinium groups, trapped by these biologicals into complexes. These complexes are formulations that can be parenterally administered. Since the binding of biological molecules with the pyridinium is non-covalent in nature, these bonds are reversible and could be engineered to have a useful rate of disassociation, so that injected large complexes of biological molecules bound to the multiple pyridinium-construct will disassociate gradually releasing the biological in a sustained release fashion. Formulations for the sustained release of small molecule drugs have proven very beneficial; comparable sustained release formulation of large biological therapeutic molecules will also have been applicable.

### Example 7. Use of Multiple Copy Pyridinium Constructs to Cross-Link Biological Molecules

Constructs having two or more pyridinium rings on a synthetic stems can be used to non-covalently link to biological molecules. The synthetic stem in these embodiments can be a relatively short hydrocarbon linker (when having two pyridinium molecules) or long hydrocarbon (synthetic or biological, like polysaccharides). Mixing the two biological molecules, including proteins, with a multiple copy pyridinium construct results in the complexing of the two biological molecules. This complexing may allow for the linking of two biological activities. As examples, the complexing of proteins having an binding specificity of interest (like an antibody) with an enzymatic protein (like horse radish peroxidase) may have diagnostic application in assays like ELISA. These approaches can serve as therapeutic treatments where the binding specificity is for a disease target and the enzymatic activity has a therapeutic effect for the disease.

Another embodiment of the invention involves the use of pyridinium ring to cross-link two biological molecules that are ligand for cellular receptor molecules, including those receptors on cell surfaces. Many cellular activation signals involve the one or more receptors on the cell surface being cross-linked by their binding to ligands on large molecule, molecular complex or physical body. The complexing of ligands by the pyridinium constructs should provide forms of these ligand that can more readily, effectively cross-linking cellular receptors and hence activating biological responses to the ligands. Pyridinium constructs could be used to non-covalently cross-link biopharmaceuticals, including antibody products. Large pyridinium constructs and complexes can be used as a shuttle or a depot for transport and release of biopharmaceuticals at targeted sites. Small pyridinium constructs can be used for transformation of biopharmaceuticals into multivalent entities and enhancing the activity by crosslinking it to the cell surface of targets or receptors.

### Example 8. Use Pyridinium Constructs to Coat Surfaces

In one embodiment of the invention, pyridinium constructs have synthetic stems that bind to the surfaces of interest for different applications. This binding of the stem to the surface may be by either covalent or non-covalent binding. The binding of the stem to the surface coats the surface with pyridinium molecules which can subsequently be treated with and bind to biological molecules by non-covalent, hydrophobic binding. This binding of the pyridinium to biological molecules coats the surfaces with those biological molecules. This coating process provides for a broad range applications including for diagnostics, industrial coating, and the coating of medical implants.

### Example 9—Use of Pyridinium-Drug Constructs for Association of Small Molecule Drugs to Biological Molecules, Including Antibodies, for the Targeted Delivery of Drugs

Drug-pyridinium constructs can be synthesized by covalently linking a small molecule drug to pyridinium, possible via the nitrogen in the aromatic ring. This drug-pyridinium construct can non-covalently, hydrophobically bind to biological molecules, including antibody proteins having a binding specificity of therapeutic interest. For example the drug in the drug-pyridinium construct could be a cytotoxic drug of tumors, and the antibody having specificity for a tumor antigen of interest. In this embodiment the toxic drug-pyridinium construct would be non-covalently bound to a tumor specific antibody and this ternary complex of drug-pyridinium-antibody would be therapeutically administered. The antibodies, upon parenteral administration, systemically circulate until it binds specifically to the tumor antigen being expressed by the tumor in the treated patient. The binding of the antibody to the tumor will deliver the cytotoxic drug specifically to the tumor like that of antibody-drug conjugates (ADC) which covalently link small molecule drugs to antibody molecules. ADC however, requires that the covalent linker attaching the drug to the antibody be engineered so that the linker can be hydrolyzed as the ADC product is internalized by the tumor cells. This is a requirement because the drug must be released from the antibody for the drug to be cytotoxic and since drug is covalently bound release of the linker must be hydrolyzed to facilitate the release. According to the invention, drug-pyridinium constructs are released from their antibody without hydrolysis because the constructs are bound to the antibodies non-covalently. This aspect of the invention therefore provides a significant advantage over standard ADC products.

### Example 10. PEGylation of Biological Molecules

PEGylation of biological molecules has proven to mediate longer half-lives and lower immunogenicity when injected into animals and human when compared to the native, non-PEGylation forms of the same biological molecules. PEGylation of biological molecules has typically required the chemical, covalent conjugation of PEG to biological molecules, including proteins. In one embodiment of the invention, a moiety which confers longer half-life and/or reduced immunogenicity, for example PEG, is covalently linked to a heterocyclic compound, an example of which is pyridinium ring systems. The PEG-pyridinium complex is then non-covalently associated with a biological molecule, thereby conferring on the biologic a longer half-life and/or reduced immunogenicity. According to the invention, pyridinium constructs with a single or multiple copies of pyridinium covalently linked to a synthetic stem which is or contains PEG, and the stem then interacts with a biological molecule to form a PEGylated biological molecules in which the PEG is non-covalently associated with the biological molecule. The pyridinium in these PEG containing constructs will bind hydrophobically to a biological molecule thereby associating PEG to the biological molecule. The biological molecules PEGylated by pyridinium constructs will have longer half-lives and be less immunogenic when administered in animals and humans.

### Example 11. Use of Pyridinium for Chromatography

Heterocyclic compounds, for example pyridinium, can be used to for chromatography media where the synthetic stem can be a chromatography matrix or be a molecular linker covalently attached to a chromatography matrix. These matrix include agarose (like that in Sepharose), dextran (like that in Sephadex), cellulose and silica. The chromatography matrices provide a solid structure from which the pyridinium, or other water miscible heterocyclic compounds, can be attached to the matrix but interact in aqueous buffers. Water soluble biological molecules run through the chromatography will interact with the pyridinium groups via their hydrophobic domains and non-covalently binding those biological molecules to the chromatography media.

These bound biological molecules, in this example biological receptors, are used to subsequently interact with other water soluble biological ligands by specific receptor ligand interactions like that between antibodies and antigens. In these applications a nonspecific biological molecule blocker, like albumin, may be used to treat the chromatography blocking free pyridinium from binding the ligand. After the binding of the receptor-ligand interactions, which are often electrostatic in nature, the ligand is eluted from the chromatography media using antichaotropic agents which disrupt the electrostatic interactions of the receptor-ligand binding but will not affect the hydrophobic binding of the pyridinium to the receptor.

Alternatively, after the binding for biological molecules to the pyridinium on the chromatography matrix, the biological molecules that were bound can be eluted using chaotropic agents which disrupt hydrophobic interactions. Chaotropic agents include:
Butanol
Ethanol
Guanidinium salts, including guanidinium chloride
Lithium perchlorate
Lithium acetate
Magnesium chloride
Phenol
Propanol
Sodium dodecyl sulfate
Thiourea
Urea

### Example 12—Quantitative Analysis of Binding of Pyridinium Constructs Using Microscale Thermophoresis

Binding affinities of pyridinium constructs with macromolecules were measure using Monolith NT.115 system developed by Nano Temper technology (https://nanotempertech.com/monolith/). The Monolith NT.115 equipment measures the strength of the interactions between a fluorescently labeled or intrinsically fluorescent sample and a binding partner such as a macromolecule are measured while a temperature gradient is applied over time. The molecular mobility of a fluorescently labeled construct in a thermo gradient when alone and in presence of increasing concentrations of binding partner is compared. From this, binding affinity (Kd) is calculated from a fitted curve that plots normalized fluorescence against concentration of ligand.

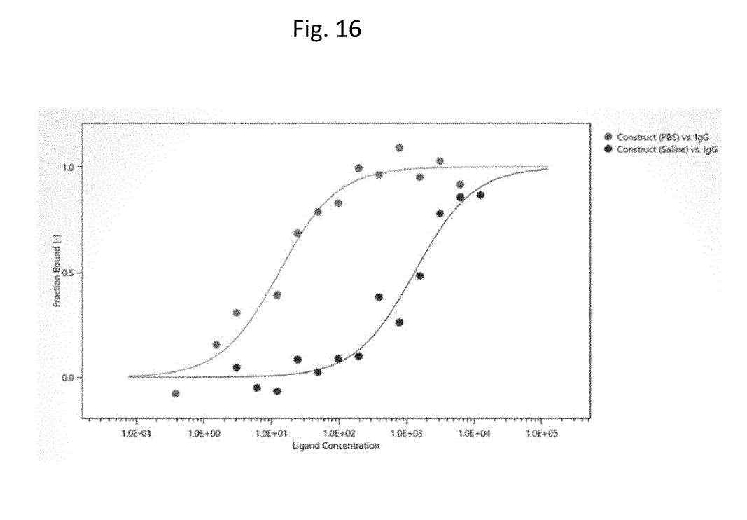

The experiment was conducted to measure the binding affinity of the pyridinium construct containing fluorescein with increasing concentrations of Immunoglobulin G (IgG) in presence of PBS buffer and saline. The results of the binding affinity curve at increasing concentrations of IgG in two different solutions (PBS & Saline) are shown in FIG. 16.

The IgG protein was tested at a concentration range of 12.5 μM-350 pM and the pyridinium-fluorescein construct was at a concentration of 500 nM. Likewise, for experiments involving HSA, the HSA protein was at a concentration range of 125 μM-3.5 nM and the pyridinium-fluorescein construct was at a concentration of 500 nM.

The pyridinium-fluorescein construct bound to IgG in both solutions but surprisingly had different binding affinities. The binding affinity (Kd) for pyridinium-fluorescein construct for IgG in PBS was 20 nm whereas the Kd for pyridinium-fluorescein construct for IgG in saline was 1.5 μM.

Without being bound by theory, it is postulated that the differences in binding affinity are primarily due to differences in nature of interactions between the pyridinium construct and the macromolecule. In presence of PBS, the nature of interactions between the construct and macromolecule are predominantly hydrophobic in nature whereas the nature of interactions between the construct and macromolecule are predominantly electrostatic in nature.

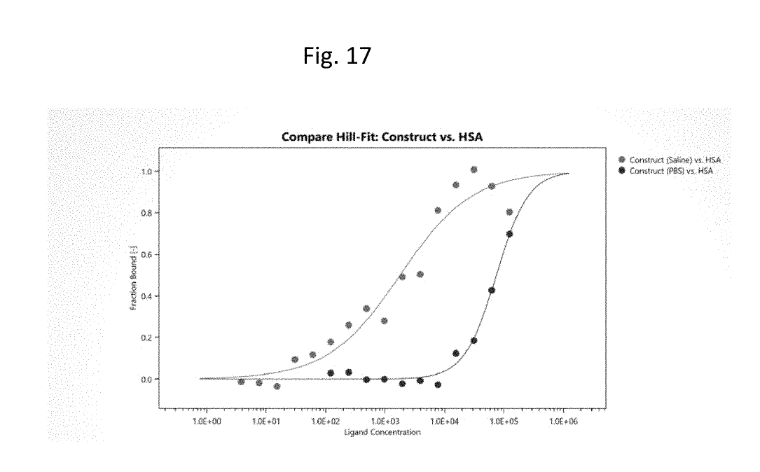

The experiment was repeated with human serum albumin (HSA) instead of IgG under similar conditions. The results of the binding affinity curve at increasing concentrations of HSA in two different solutions (PBS & Saline) are shown in FIG. 17. The results showed that the binding affinity of pyridinium construct to HSA was surprisingly comparable to that of IgG under saline solution. However interestingly the binding affinity of pyridinium construct to HSA was much lower than that of IgG under PBS solution.

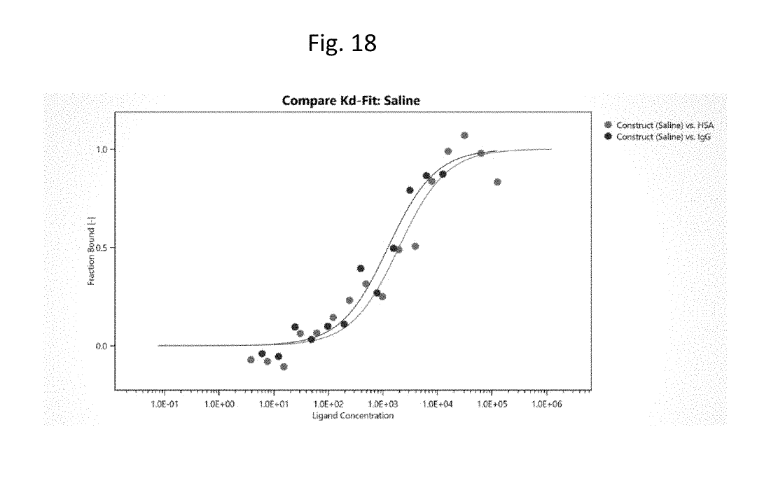

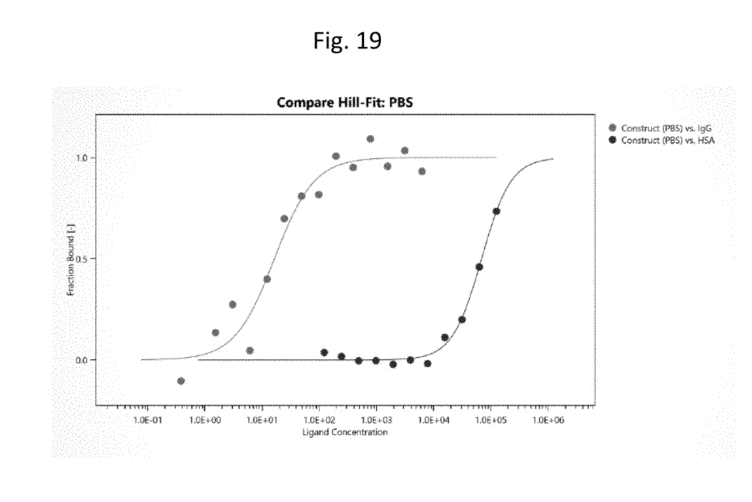

FIG. 18 shows the comparative binding affinity curves for pyridinium constructs with IgG and HS under saline solution. FIG. 19 shows the comparative binding affinity curves for pyridinium constructs with IgG and HSA under PBS solution.

Without being bound by theory, it is postulated that the binding affinities of the pyridinium constructs to HSA and IgG are similar under saline conditions because of similar electrostatic binding potentials. The pI of HSA is 5.6 and pI of IgG is 6.5, both proteins are negatively charged under physiological conditions in saline solutions whereas the pyridinium construct is positively charged. Hence the apparent electrostatic binding to both proteins is roughly of the same affinity.

However in case of PBS solution, the binding affinity is predominantly hydrophobic in nature. The data indicates that hydrophobic interaction of the pyridinium construct is higher in IgG under PBS. This implies that there are strong pi-pi interactions between the pyridinium construct and the protein residues of IgG. High binding affinity of pyridinium constructs to IgG would be desirable in certain pharmaceutical applications wherein immunoglobulin based biological drugs are being utilized to treat diseases. One possible application could be to link two molecules of IgG using pyridinium constructs to increase activity or to even attach chemotoxic drugs to IgG through pyridinium constructs to enhance their activity.

### Example 13—Synthesis of Dextran-Pyridinium Constructs

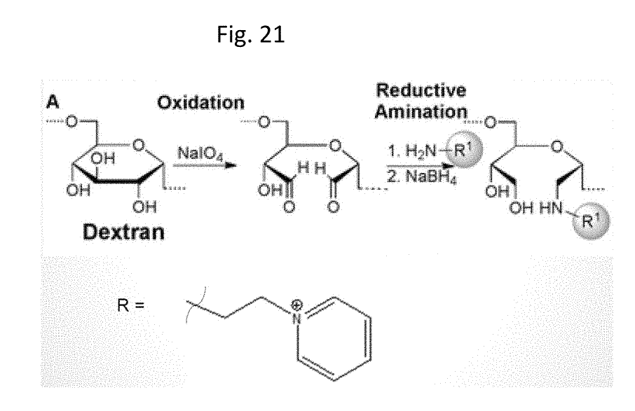

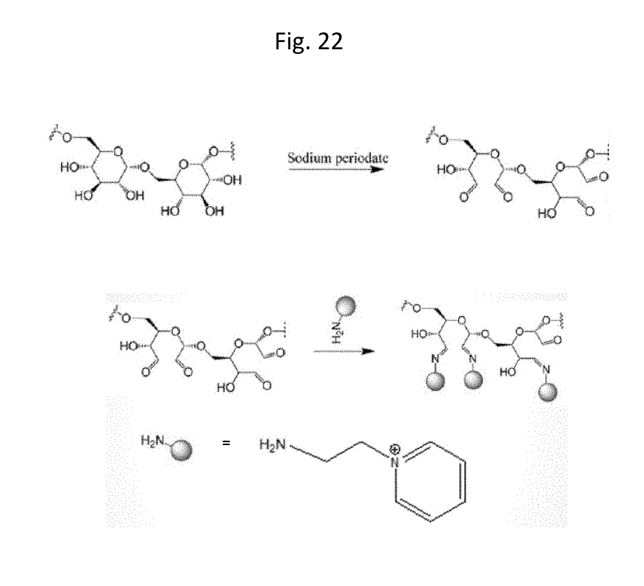

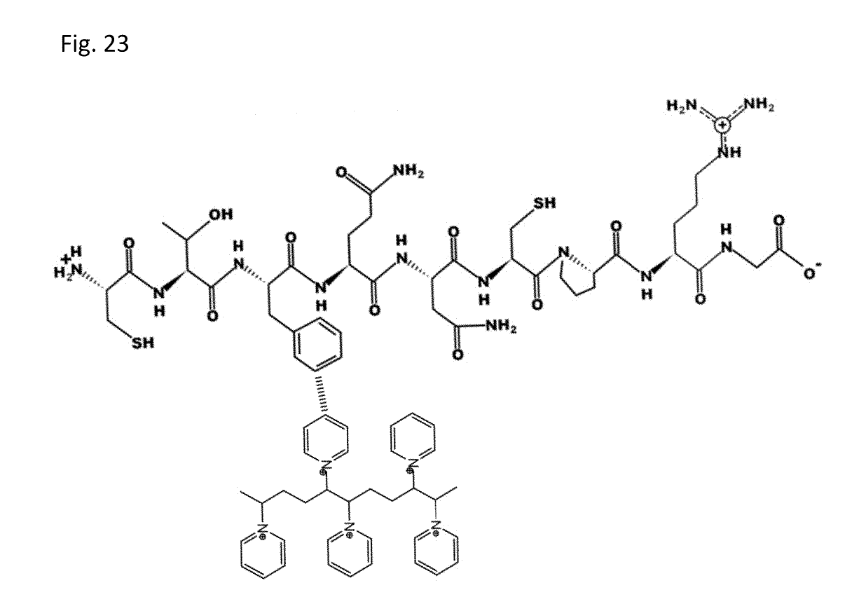

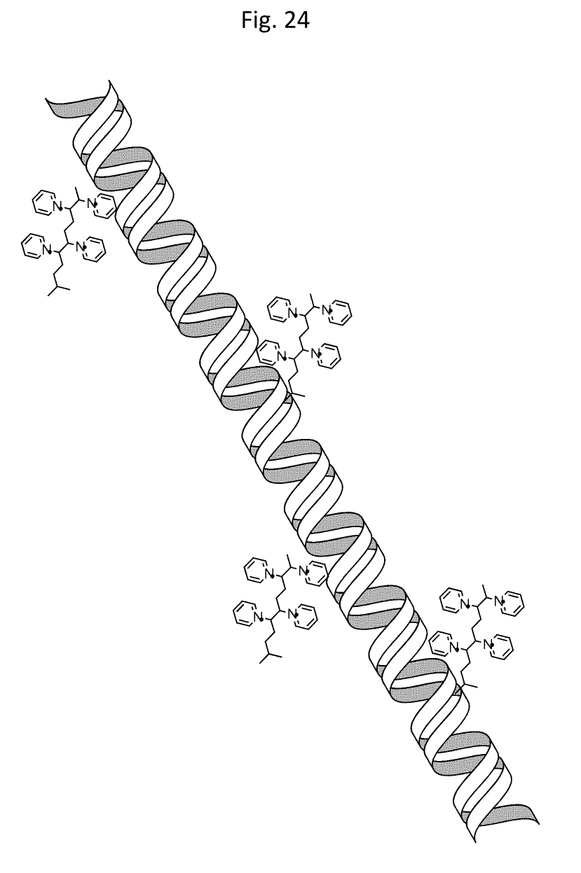

The following example details the synthetic process utilized in the production of chemical composition comprising a heterocyclic pyridinium ring, synthetic stem and dextran as shown in FIGS. 21 and 22. One of skill in the art would understand that this synthetic process can be optionally modified using knowledge in art to produce chemical formulations comprising multiple heterocyclic rings and multiple carbohydrate moieties.

The synthesis of Dextran-Pyridinium constructs is carried out in two steps. The first step involves periodate oxidation of dextran and the reductive amination of oxidized dextran with pyridinium amine resulting in the formation of Dextran-pyridinium constructs. The process can repeated with dextrans of different molecular weight ranging from about 1 KDa to about 500,000 KDa, preferably from about 10 KDa to about 100,000 KDa, more preferably from about 5 KDa to about 100 KDa. The amount of oxidation in dextran can be varied by changing the concentration of periodate. The oxidations in dextran can range from about 5% to about 100% oxidation, preferably from about 5% to about 50% oxidation, more preferably from about 5% to about 25% oxidation. The amount of pyridinium rings incorporated in the dextran constructs can also be varied by changing the concentration of pyridinium amine. The pyridinium incorporations in the dextran can range from about 1% to about 100%, preferably from about 5% to about 50%, more preferably from about 5% to about 25% pyridinium incorporation.

The first step of periodate oxidation of dextran as shown in FIG. 21 is carried out as follows. An aqueous solution of dextran (6 kDa; ˜33 residues; 1 g in 7 mL or ˜15% weight/volume; ˜23 mmol) was oxidized with 2 mL of sodium periodate solution with varied concentrations to yield theoretical oxidations of 5%, 10%, and 20% at room temperature. The reaction was stopped after four hours. The resulting solution was dialyzed for three days against water and then lyophilized as described in J Maia et al, Polymer 46 (2005), 9604-9614. The contents of which are fully incorporated by reference in its entirety.

The second step of reductive amination with oxidized dextran and pyridinium amine as shown in FIG. 22 is carried out as follows. The illustrated example uses 5% oxidized dextran but the process is equally applicable for other ranges of oxidized dextran and varying molecular weight dextrans as well and hence should not be construed to be limiting the invention in anyway.

50 mg of 5% oxidized 6 kDa dextran (˜270 mmol) was dissolved in 1 mL dimethyl sulfoxide (DMSO) and then 60 mg (˜290 mM; 1:1 equivalence) pyridinium was added to the solution. It was followed up with several of glacial acetic acid to acidify the solution. The reaction was allowed to stir at room temperature for two hours in order to facilitate the formation of imine. Following the formation of imine, 100 mg of sodium triacetoxyborohydride was added (˜470 mmol, 1.7 equivalence). The reaction was stirred overnight and the completed reaction was purified via dialysis in water overnight as described in Abdel-Magid, K G Carson, B. D Harris, C. A Maryanoff, R. D Shah, J Org. Chem., 1996, 61, 3849-3862. The contents of which are fully incorporated by reference in its entirety.

The Dextran-pyridinium constructs thus produced have the ability to form complexes with antibody or biopharmaceuticals and can release the antibody or biopharmaceuticals over time thereby serving as extended release formulations.

### Example 14—Quantitative Analysis of Binding of Pyridinium Constructs with Peptides

This example illustrates the interactions between the pyridinium constructs containing biotin and a peptide. Nonlimiting examples of a peptide includes but is not limited to: vassopressin, bradykinin, colivellin, mellitin, neuromedin, neurotensin etc. The pyridinium construct containing biotin and the pyridinium construct containing fluorescein is synthesized and purified as described in Example 1

Surface Plasmon Resonance (SPR) can be used to quantitate the binding of pyridinium constructs containing biotin to a peptide as described in Example 3. Briefly through a microflow system, a solution with a peptide of interest (0.001 to 10 ug/ml) is injected over the pyridinium construct covered sensor chip surface. As the peptide binds to the pyridinium constructs, an increase in SPR signal (expressed in response units, RU) is observed. After desired association time, a solution without the peptide (usually buffer containing antichaotropic agents) is injected on the microfluidics that will enable the dissociation of the bound complex between pyridinium construct and peptide. The dissociation of complex between the pyridinium construct and peptide ligand will result in a decrease in SPR signal (expressed in resonance units, RU). From these association (‘on rate’, ka) and dissociation rates (‘off rate’, kd), the equilibrium dissociation constant (‘binding constant’, KD) can be calculated. (KD=kd/ka).

Without being bound by theory, it is postulated that pyridinium constructs and other heterocyclic constructs as exemplified in FIG. 8 would predominantly form hydrophobic interactions with the peptide residues under suitable buffer conditions such as PBS. It is expected that the interactions between the heterocyclic ring systems and the peptides would become predominantly electrostatic when the molecules become more polarized with changes in the pH which is in turn is connected with the isoelectric point of the peptide entities.

Binding analysis can also be performed using fluorescent pyridinium constructs and peptides by employing microscale thermophoresis techniques as described in Examples 2 and 12.

### Example 15—Quantitative Analysis of Binding of Pyridinium Constructs with Nucleic Acids

This example illustrates the interactions between the pyridinium constructs containing biotin and a nucleic acid. Nonlimiting examples of nucleic acids include but are not limited to PNA, DNA, RNA, cDNA, single stranded oligonucleotides, plasmids, double stranded nucleotides, hairpin loop structures and nucleotide duplexes with 3′ or 5′ overhangs. The pyridinium construct containing biotin and the pyridinium construct containing fluorescein is synthesized and purified as described in Example 1

Surface Plasmon Resonance (SPR) can be used to quantitate the binding of pyridinium constructs containing biotin to a nucleic acid as described in Example 3. Briefly through a microflow system, a solution with a nucleic acid of interest (0.001 to 10 ug/ml) is injected over the pyridinium construct covered sensor chip surface. As the nucleic acid binds to the pyridinium constructs, an increase in SPR signal (expressed in response units, RU) is observed. After desired association time, a solution without the nucleic acid (usually buffer containing antichaotropic agents) is injected on the microfluidics that will enable the dissociation of the bound complex between pyridinium construct and nucleic acids. The dissociation of complex between the pyridinium construct and nucleic acid ligand will result in a decrease in SPR signal (expressed in resonance units, RU). From these association (‘on rate’, ka) and dissociation rates (‘off rate’, kd), the equilibrium dissociation constant (‘binding constant’, KD) can be calculated. (KD=kd/ka).

Without being bound by theory, it is postulated that pyridinium constructs and other heterocyclic constructs as exemplified in FIG. 8 would predominantly form hydrophobic interactions with the nucleic acids by intercalating between bases in the hydrophobic pockets created by Pi-Pi stacking interactions under suitable buffer conditions such as PBS. It is expected that the interactions between the heterocyclic ring systems and the nucleic acids would become predominantly electrostatic when the nucleic acids become more polarized with changes in the pH.

Binding analysis can also be performed using fluorescent pyridinium constructs and nucleic acids by employing microscale thermophoresis techniques as described in Examples 2 and 12.

### Other Embodiments

From the foregoing description, it will be apparent that variations and modifications may be made to the invention described herein to adopt it to various usages and conditions, including the use of other heterocyclic and heterocyclic aromatic structures other than pyridinium. Such embodiments are also within the scope of the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

What is claimed is:

1. A composition comprising a heterocyclic ring, a synthetic stem, and a biological molecule wherein the heterocyclic ring is (a) covalently linked to the synthetic stem and (b) non-covalently associated with the biological molecule.

2. The composition of claim 1, wherein said non-covalent association is stable upon exposure to antichaotropic salts.

3. The composition of claim 2 wherein said non-covalent association comprises hydrophobic interactions.

4. The composition of claim 3, wherein said hydrophobic interactions are selected from the group consisting of pi interactions, pi-pi interactions and pi stacking interactions.

5. The composition of claim 1, wherein said synthetic stem comprises a substituted or un-substituted hydrocarbon.

6. The composition of claim 1, wherein said synthetic stem comprises a small molecule drug.

7. The composition of claim 1, wherein said synthetic stem comprises PEG.

8. The composition of claim 1, wherein said heterocyclic ring comprises a heterocyclic aromatic quaternary amine.

9. The composition of claim 1, wherein said heterocyclic ring is water miscible.

10. The composition of claim 8, wherein said heterocyclic ring comprises a pyridinium ring.

11. The composition of claim 9, wherein said heterocyclic ring is positively charged over a pH range of about 3 to about 10.

12. The composition of claim 1, wherein said biological molecule is selected from a group consisting of antibodies, proteins, peptides, DNA, RNA and DNA ligands.

13. The composition of claim 1, wherein more than one copy of the heterocyclic ring is covalently linked to said synthetic stem.

14. The composition of claim 12, wherein said heterocyclic rings are covalently linked to said synthetic stem and are non-covalently associated with the same or different biological molecules, linking multiple copies of the biological molecules to the same synthetic stem.

15. The composition of claim 13, wherein said synthetic stem is covalently or non-covalently associated with molecules in a non-aqueous phase, and the heterocyclic rings are non-covalently associated with the same or different biological molecules in an aqueous phase.

16. The composition of claim 12, wherein said non-covalent association is reversible or partially reversible under physiological conditions.

17. The composition of claim 12, wherein said synthetic stem is covalently attached to said small molecule drug and said heterocyclic rings are non-covalently associated with the same or different biological molecules.

18. The composition of claim 12, wherein said synthetic stem is covalently linked to a small molecule drug or a macromolecular drug, said heterocyclic rings are non-covalently associated to said biological molecules, wherein said biological molecules are of same kind or different from one another.

19. A method of treating cancer or an auto-immune disease, the method comprising administering a therapeutically effective amount of a composition comprising a heterocyclic ring, a synthetic stem comprising a small molecule drug or a macromolecular drug known to be used to treat said disease, and a biological molecule wherein the heterocyclic ring is (a) covalently linked to the synthetic stem and (b) non-covalently associated with the biological molecule.

20. The method of claim 19, wherein said synthetic stem is covalently attached to said small molecule drug or said macromolecular drug and said heterocyclic rings are non-covalently associated with the same or different biological molecules.

21. The method of claim 19, wherein said biological molecule is a drug known to be used to treat said disease.

22. A composition comprising a heterocyclic ring, a synthetic stem, and a biological molecule wherein the heterocyclic ring is (a) covalently linked to the synthetic stem and (b) non-covalently associated with the biological molecule such that said biologic molecule is stably associated with said heterocyclic ring upon exposure to antichaotropic salts.

23. A composition comprising a heterocyclic ring comprising a heterocyclic aromatic quaternary amine or a pyridinium ring, a synthetic stem comprising a substituted or un-substituted hydrocarbon, and a biological molecule, wherein the heterocyclic ring is (a) covalently linked to the synthetic stem and (b) non-covalently associated with the biological molecule via hydrophobic interactions.


SEQUENCE LISTING (1 sequence)

SEQ ID NO: 1 - protein, 9 aa, artificial
  synthetic representative peptide vasopressin
CYFQNCPRG